(19) United States
(12) Patent Application Publication
(10) Pub. No.: US 2023/0287132 A1
(43) Pub. Date: Sep. 14, 2023

(54) METHODS OF TREATING OR REDUCING RISK OF TRANSPLANT REJECTION

(71) Applicants: Kiniksa Pharmaceuticals, Ltd., Hamilton (BM); University of Maryland, Baltimore, Baltimore, MD (US)

(72) Inventors: John F. PAOLINI, Lexington, MA (US); Muhammad M. MOHIUDDIN, Baltimore, MD (US)

(21) Appl. No.: 18/152,519

(22) Filed: Jan. 10, 2023

Related U.S. Application Data
(60) Provisional application No. 63/327,986, filed on Apr. 6, 2022.
(60) Provisional application No. 63/307,911, filed on Feb. 8, 2022.
(60) Provisional application No. 63/298,133, filed on Jan. 10, 2022.

(51) Int. Cl. C07K 16/28; C07K 16/18; A61K 45/06; A61P 37/06

(52) U.S. Cl. CPC C07K 16/2878; C07K 16/2887; C07K 16/18; A61K 45/06; A61P 37/06; A61K 2039/545

(57) ABSTRACT

The present disclosure relates to methods of treating or reducing the risk of transplant rejection or increasing a duration of time before transplant rejection occurs in a subject in need thereof (e.g., a human) by administering an antagonist that targets CD40 or CD154, such as an anti-CD40 antibody or an antigen-binding fragment thereof (e.g., a humanized anti-CD40 antibody or antigen-binding fragment thereof. The transplant may be an allogeneic or xenogeneic transplant (e.g., a cell, tissue, or organ or portion thereof).

### SEQUENCE LISTING

This application contains a Sequence Listing which has been filed electronically in XML format and is hereby incorporated by reference in its entirety. Said XML copy, created on Dec. 29, 2022, is named 51383-008004_Sequence_Listing_12_29_22.XML and is 10,397 bytes in size.

### BACKGROUND

Tissue and organ transplantation is a powerful therapeutic tool for replacing a damaged or failing organ or tissue in a subject in need thereof. However, a major problem following transplantation occurs when the recipient's immune system attacks the transplant, leading to rejection and failure of the transplant. There is a critical shortage of human tissues and organs for the purposes of tissue and organ transplantation.

Xenotransplantation, which is the transplantation of an organ or tissue from a species other than human into a human recipient, could help to address the shortage of human donor material. Xenotransplantation presents significant obstacles, as certain antigens on a non-human donor organ can elicit an immune response by the human recipient, leading to acute rejection of the transplanted material.

Accordingly, new methods are needed to suppress the immune system to treat or reduce the risk or likelihood of transplant rejection or to increase the time before transplant rejection occurs in a human subject that receives a transplant (e.g., an allogeneic or xenogeneic transplant).

### SUMMARY

A first aspect features a method of treating or reducing the risk of transplant rejection or increasing a duration of time before transplant rejection occurs in a human subject receiving or having received a transplant (e.g., a xenotransplant). The method includes administering an anti-CD40 antibody or antigen-binding fragment thereof from about 1 hour to about 24 hours (e.g., from about 3 hours to about 24 hours, or about 10 hours to about 24 hours) prior to the transplant and administering the anti-CD40 antibody or antigen-binding fragment thereof within about 1 hour to about 24 hours following the transplant, for example, once the subject achieves hemostasis. In certain embodiments, the method treats, reduces the risk of, or increases the duration of time before the occurrence of, hyperacute rejection, acute rejection, or chronic rejection of the transplant. The antibody or antigen-binding fragment thereof may be administered subcutaneously or intravenously.

In some embodiments, the antibody or antigen-binding fragment thereof includes a heavy chain variable region including a CDRH1, CDRH2, and CDRH3 as set forth SEQ ID NOs: 1-3, respectively, and a light chain variable region including a CDRL1, CDRL2, and CDRL3 as set forth in SEQ ID NOs: 4-6, respectively. In some embodiments, the heavy chain variable region includes an amino acid sequence having at least 85% (e.g., at least 90%, 95%, 97%, 99%, or 100%) sequence identity to SEQ ID NO: 7, and the light chain variable region includes an amino acid sequence having at least 85% (e.g., at least 90%, 95%, 97%, 99%, or 100%) sequence identity to SEQ ID NO: 8. In some embodiments, the heavy chain variable region includes the amino acid sequence set forth in SEQ ID NO: 7, and the light chain variable region includes the amino acid sequence set forth in SEQ ID NO: 8. In some embodiments, the heavy chain includes the amino acid sequence set forth in SEQ ID NO: 9, and the light chain includes the amino acid sequence set forth in SEQ ID NO: 10.

In some embodiments, the antibody or antigen-binding fragment thereof is administered at a dosage of about 1 mg/kg to about 20 mg/kg (e.g., a dosage of about 5 mg/kg or about 10 mg/kg).

A second aspect features a method of treating or reducing the risk of transplant rejection or increasing a duration of time before transplant rejection occurs in a human subject receiving or having received a transplant (e.g., a xenotransplant). The method includes administering an anti-CD40 antibody or antigen-binding fragment (e.g., a humanized anti-CD40 antibody or antigen-binding fragment). In certain embodiments, the method treats, reduces the risk of, or increases the duration of time before the occurrence of hyperacute rejection, acute rejection, and/or chronic rejection of the transplant. The antibody or antigen-binding fragment thereof includes a heavy chain variable region with a CDRH1, CDRH2, and CDRH3 as set forth SEQ ID NOs: 1-3, respectively, and a light chain variable region with a CDRL1, CDRL2, and CDRL3 as set forth in SEQ ID NOs: 4-6, respectively. The antibody or antigen-binding fragment thereof may be administered subcutaneously or intravenously, e.g., at a dosage of about 1 mg/kg to about 20 mg/kg (e.g., a dosage of about 10 mg/kg).

In some embodiments, the heavy chain variable region includes an amino acid sequence having at least 85% (e.g., at least 90%, 95%, 97%, 99%, or 100%) sequence identity to SEQ ID NO: 7, and the light chain variable region includes an amino acid sequence having at least 85% (e.g., at least 90%, 95%, 97%, 99%, or 100%) sequence identity to SEQ ID NO: 8. In some embodiments, the heavy chain variable region includes the amino acid sequence set forth in SEQ ID NO: 7, and the light chain variable region includes the amino acid sequence set forth in SEQ ID NO: 8. In some embodiments, the heavy chain includes the amino acid sequence set forth in SEQ ID NO: 9, and the light chain includes the amino acid sequence set forth in SEQ ID NO: 10.

In some embodiments, the antibody or antigen-binding fragment thereof is administered prior to the transplant (e.g., from about 1 hour to about 24 hours, e.g., from about 10 hours to about 24 hours prior to the transplant, e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant).

In some embodiments, the antibody or antigen-binding fragment thereof is administered on the same day as the transplant.

In some embodiments, the antibody or antigen-binding fragment thereof is administered after the transplant.

In some embodiments, the antibody or antigen-binding fragment thereof is administered within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant, for example, once the subject achieves hemostasis.

In some embodiments, the antibody or antigen-binding fragment thereof is administered one day, two days, three days, four days, five days, six days, one week, two weeks, three weeks, four weeks, two months, three months, four months, five months, six months, seven months, eight months, nine months, ten months, 11 months, and/or one year after the transplant.

In some embodiments, the antibody or antigen-binding fragment thereof is administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, the antibody or antigen-binding fragment thereof is administered at least once every week, once every two weeks, once every three weeks, or once a month for a treatment period of one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, about 1 mg/kg to about 20 mg/kg (e.g., about 5 mg/kg or about 10 mg/kg) of the antibody or antigen-binding fragment thereof is administered at least once every week, once every two weeks, once every three weeks, or once a month for a treatment period of one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, the method includes administering the antibody or antigen-binding fragment thereof as an induction dose that includes two doses: a first dose of about 1 mg/kg to about 20 mg/kg (e.g., a dose of about 10 mg/kg) that is administered prior to the transplant (e.g., from about 1 hour to about 24 hours, e.g., from about 10 hours to about 24 hours prior to the transplant (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant) and a second dose of about 1 mg/kg to about 20 mg/kg (e.g., a dosage of about 10 mg/kg) that is administered within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant (e.g., once the subject achieves hemostasis). In one embodiment, the second dose is administered once the subject achieves hemostasis. In certain aspects of the invention, the induction dose administered prior to the transplant is a “conditioning dose” that functions to prophylactically suppress or modulate the subject's immune system with an anti-CD40 antibody or antigen-binding fragment thereof prior to the transplant. The method further includes treating the subject with subsequent maintenance dosing of the antibody or antigen-binding fragment thereof (e.g., a dose of the antibody or antigen-binding fragment thereof that follows after the induction dose) of about 1 mg/kg to about 20 mg/kg (e.g., a dose of about 10 mg/kg) that is administered once every week, once every two weeks, once every three weeks, or once a month (e.g., once every two or three weeks). In certain embodiments, maintenance dosing is initiated once the anti-CD40 antibody or antigen-binding fragment thereof has replicable (e.g., within a target therapeutic range) or predictable pharmacokinetics (PK) in the subject. The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject).

In some embodiments, an induction dose includes a single, undivided dose of the antibody or antigen-binding fragment thereof. Alternatively, an induction dose may include a divided dose of the antibody or antigen-binding fragment thereof that includes at least two doses. For example, in some embodiments, an induction dose is a divided dose that includes a first dose and a second dose of the antibody or antigen-binding fragment thereof that are administered about 1 hour to about 24 hours apart, e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours apart, such as from about 1 to about 12 hours apart). For example, the first divided dose of the induction dose may involve administration of from about 1 mg/kg to about 20 mg/kg of the antibody or antigen-binding fragment thereof (e.g., a dosage of about 10 mg/kg) and a second divided dose of the induction dose may involve administration of from about 1 mg/kg to about 20 mg/kg of the antibody or antigen-binding fragment thereof (e.g., a dosage of about 10 mg/kg).

The second divided dose of the induction dose may be administered about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours, such as from about 1 to about 12 hours) after the subject receives the first divided dose of the induction dose. For example, to the method may involve administration of a divided dose of an induction dose of 20 mg/kg of the anti-CD40 antibody or antigen-binding fragment thereof, in which the first divided dose provides about 10 mg/kg of the antibody or antigen-binding fragment thereof and the second divided dose provides about 10 mg/kg of the antibody or antigen-binding fragment thereof.

The method may include administration of multiple induction doses (e.g., either as undivided doses or as divided doses). For example, in some embodiments, the method includes administering 1, 2, 3, 4, 5, 6, 7, 8, 9, 10, or more induction doses (e.g., from about 1 to about 5 induction doses such as, e.g., about 3 or about 4 induction doses). In some embodiments, each induction dose (e.g., first induction dose, second induction dose, third induction dose, fourth induction dose, or more) is a divided dose that includes first and second doses of the antibody or antigen-binding fragment thereof (e.g., each of the first or second dose in an amount of about 1 mg/kg to about 20 mg/kg per dose) that are, e.g., administered between 1 and 24 hours apart (e.g., 1 to 12 hours apart)). Alternatively, the first induction dose is a divided dose and any additional induction dose(s) (e.g., first, second, third, fourth, or more induction dose(s)) is/are administered as either a divided dose of the antibody or antigen-binding fragment thereof (e.g., a divided dose of at least two doses that are administered in an amount of about 1 mg/kg to about 20 mg/kg per dose, in which the doses are, e.g., administered between 1 and 24 hours apart (e.g., 1 to 12 hours apart)) or an undivided dose of the antibody or antigen-binding fragment thereof (e.g., a single dose in an amount of about 1 mg/kg to about 20 mg/kg). In another embodiment, the first induction dose is a divided dose (e.g., a divided dose of at least two doses that are administered in an amount of about 1 mg/kg to about 20 mg/kg per dose, in which the doses are, e.g., administered between 1 and 24 hours apart (e.g., 1 to 12 hours apart)) and any additional induction dose(s) (e.g., first, second, third, fourth, or more induction dose(s)) is/are administered as a divided dose (e.g., including at least two doses in an amount of about 1 mg/kg to about 20 mg/kg per dose). In some embodiments, a second induction dose is administered from about 1 day to about 14 days (e.g., 1 day, 2 days, 3 days, 4 days, 5 days, 6 days, 7 days, 8 days, 9 days, 10 days, 11 days, 12 days, 13 days, or 14 days) following administration of the first induction dose. In other embodiments, second, third, fourth, and/or fifth induction doses are administered from about 1 day to about 14 days apart (e.g., 1 day, 2 days, 3 days, 4 days, 5 days, 6 days, 7 days, 8 days, 9 days, 10 days, 11 days, 12 days, 13 days, or 14 days apart) and, optionally, within 1 to 30 days after the first induction dose.

In some embodiments, the induction dose is provided to attain or maintain a desired minimum blood, plasma, or serum concentration of the antibody or antigen-binding fragment thereof. For example, in some embodiments, it is desirable to maintain a minimum concentration (e.g., a trough concentration) of at least about 20 μg/mL (e.g., at least 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL (e.g., a range from about 20 ug/mL to about 150 ug/mL; from about 150 μg/mL to about 300 μg/mL; from about 150 μg/mL to about 200 μg/mL, or from about 20 μg/mL to about 300 μg/mL) of the antibody or antigen-binding fragment thereof in the blood, plasma, or serum. Accordingly, the method may include administering an induction dose and monitoring the concentration of the antibody or antigen-binding fragment thereof in the blood, plasma, or serum over time. Once the concentration approaches the minimum threshold (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, or about 150 μg/mL (or a concentration in the range of from about 20 μg/mL to about 150 μg/mL), e.g., within about 10%, 20%, 30%, 40%, or 50% of the minimum threshold), the method may include administering a further induction dose (e.g., prior to establishment of maintenance dosing e.g., at a dosage of about 1 mg/kg to about 20 mg/kg, e.g., about 5 mg/kg or about 10 mg/kg) of the antibody or antigen-binding fragment thereof.

In some embodiments, the subject is administered one of more induction doses until the PK of the anti-CD40 antibody, or antigen-binding fragment thereof, in the subject's blood, plasma, or serum (e.g., peak and trough concentrations) is replicable or is predictable (e.g., at the desired therapeutic range, such as when the C_{trough} and/or Cₘₐₓ levels appear consistent between doses). For example, in some embodiments, multiple induction doses are administered (as described herein) until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL).

In other embodiments, multiple induction doses (as described herein) are administered until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be about 20%, e.g., within 15%, 10%, 5% or less, of each other.

In another embodiment, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL)

In other embodiments, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be about 20%, e.g., within 15%, 10%, 5% or less, of each other. Optionally, the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week).

Once the PK of the antibody or antigen-binding fragment thereof in the subject's blood, plasma, or serum is replicable or predictable at a desired therapeutic range, the subject may transition from induction dosing to maintenance dosing. In certain embodiments, the subject transitions from induction dosing to maintenance dosing when (i) two or more C_{trough} of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other); and/or (ii) two or more Cₘₐₓ of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other).

Optionally, the C_{trough} and/or the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days, or once per week).

In some embodiments, the maintenance dose is provided to maintain a desired minimum blood, plasma, or serum concentration of the antibody or antigen-binding fragment thereof. For example, in some embodiments, it is desirable to maintain a minimum concentration (e.g., a trough concentration) of at least about 20 μg/mL (e.g., at least 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL (e.g., a range from about 20 ug/mL to about 150 ug/mL; from about 150 μg/mL to about 300 μg/mL; from about 150 μg/mL to about 200 μg/mL, or from about 20 μg/mL to about 300 μg/mL) of the antibody or antigen-binding fragment thereof in the blood, plasma, or serum of the subject. Accordingly, the method may include administering an induction or maintenance dose and determining the concentration of the antibody or antigen-binding fragment thereof in the subject's blood, plasma, or serum (e.g., the concentration is determined once per day, once every two days, once every three days, once every four days, once every five days, once every six days or once per week). Once the concentration approaches the minimum threshold (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, or about 150 μg/mL (or a concentration in the range of from about 20 μg/mL to about 150 μg/mL), e.g., within about 10%, 20%, 30%, 40%, or 50% of the minimum threshold), the method may include administering a maintenance dose (e.g., at a dosage of about 1 mg/kg to about 20 mg/kg, e.g., about 5 mg/kg or about 10 mg/kg) of the antibody or antigen-binding fragment thereof. These maintenance doses can be repeatedly administered following the transplant, e.g., once every week, once every two weeks, once every three weeks, or once a month (e.g., once every two or three weeks), or as needed to maintain in the subject's blood, plasma, or serum a concentration of the antibody, or antigen-binding fragment thereof, above 150 μg/ml. The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject). The timing of administration of the maintenance dose and/or the timing between maintenance doses may be selected based on the PK profile of the antibody or antigen-binding fragment thereof in, e.g., the blood, plasma, or serum of the subject. The method further includes treating the subject with a maintenance dose of the antibody or antigen-binding fragment thereof (e.g., after the final induction dose) of about 1 mg/kg to about 20 mg/kg, or about 5 mg/kg to about 10mg/kg (e.g., a dose of about 5mg/kg or about 10 mg/kg) that is administered once every week, once every two weeks, once every three weeks, or once a month (e.g., once every two or three weeks). The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject).

In some embodiments, the method further includes including administering a first therapeutic agent that depletes or reduces B cells in the subject (e.g., an anti-CD20 antibody, such as rituximab) and/or a second therapeutic agent that depletes or reduces T cells in the subject (e.g., anti-thymocyte globulin, e.g., THYMOGLOBULIN® or ATGAM®, or an anti-IL-2Rα receptor antibody).

The first therapeutic agent may be administered prior to the transplant (e.g., from about 10 hours to about 24, e.g., about 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant). The first therapeutic agent may be administered after the transplant (e.g., one day, two days, three days, four days, five days, six days, one week, two weeks, three weeks, and/or four weeks after the transplant).

In some embodiments, the first therapeutic agent is administered for a treatment period of one day, two days, three days, four days, five days, six days, one week, two weeks, three weeks, or four weeks. In some embodiments, the first therapeutic agent is administered daily, weekly, or once every other week for a treatment period of one week, two weeks, three weeks, or four weeks.

In some embodiments, the first therapeutic agent (e.g., an anti-CD20 antibody, such as rituximab) is administered at a dosage of about 1 mg/kg to about 40 mg/kg (e.g., about 10 mg/kg). In some embodiments, the first therapeutic agent is administered intravenously.

In some embodiments, the anti-CD20 antibody (e.g., rituximab) is administered at a dosage of about 250 mg/m² to about 500 mg/m², e.g., a dosage of about 375 mg/m².

In some embodiments, the first therapeutic agent is rituximab.

In some embodiments, the second therapeutic agent may be administered prior to the transplant (e.g., from about 10 hours to about 24 hours prior to the transplant, e.g., about 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant).

In some embodiments, the second therapeutic agent is administered on the same day as the transplant.

The second therapeutic agent may be administered after the transplant (e.g., one day, two days, three days, four days, five days, six days, one week, two weeks, three weeks, and/or four weeks after the transplant). In some embodiments, the second therapeutic agent is administered for a treatment period of one day, two days, three days, four days, five days, six days, one week, two weeks, three weeks, or four weeks. In some embodiments, the second therapeutic agent is administered daily, weekly, or once every other week for a treatment period of one week, two weeks, three weeks, or four weeks.

In some embodiments, the second therapeutic agent is administered within about 1 hour to about 24 hours following the transplant and once the subject achieves hemostasis).

In some embodiments, the second therapeutic agent (e.g., anti-thymocyte globulin, e.g., THYMOGLOBULIN® or ATGAM®, or anti-IL-2Rα receptor antibody) is administered at a dosage of about 1 mg/kg to about 10 mg/kg, e.g., about 2 mg/kg or about 5 mg/kg. In some embodiments, the second therapeutic agent is THYMOGLOBULIN® and is administered at a dosage of about 2 mg/kg. In some embodiments, the second therapeutic agent is ATGAM® and is administered at a dosage of about 5 mg/kg. The second therapeutic agent may be administered intravenously.

In some embodiments, the second therapeutic agent is anti-thymocyte globulin.

In some embodiments, the antibody or antigen-binding fragment thereof is administered at a dosage of about 10 mg/kg.

In some embodiments of any of the aspects described herein, the antibody or antigen-binding fragment thereof is administered intravenously.

In some embodiments of any of the aspects described herein, the antibody or antigen-binding fragment thereof is administered subcutaneously.

In some embodiments of any of the aspects described herein, the transplant is an allogeneic transplant. In other embodiments of any of the aspects described herein, the transplant is a xenograft transplant. The transplant may be a cell, tissue, or organ transplant. The transplant may be, or may include, for example, a heart, kidney, lung, liver, pancreas, intestine, thymus, skin, eye, uterus, stem cell, bone, tendon, cornea, heart valve, nerve, vein, or a portion thereof. In some embodiments, the xenograft transplant includes an organ or portion thereof from a pig, cow, horse, dog, cat, sheep, goat, non-human primate (e.g., a macaque (e.g., a rhesus macaque or a cynomolgus macaque), a baboon, a marmoset, a monkey, and a chimpanzee), or gorilla. The xenograft transplant may include a porcine heart. The xenograft transplant may include a porcine kidney. In some embodiments, the xenograft transplant has been genetically engineered, e.g., to add, modify, or remove one or more xenoreactive antigens. In some embodiments, the xenograft transplant includes an organ or portion thereof from a host animal that has been genetically engineered, e.g., to add, modify, or remove one or more xenoreactive antigens.

In some embodiments of any of the above aspects, the method further includes administering one or more of a steroid (e.g., methylprednisolone), an antihistamine (e.g., diphenhydramine), an H2 receptor blocker (e.g., famotidine), an antiviral agent (e.g., ganciclovir), a complement inhibitor (e.g., a C1 esterase inhibitor), an immunosuppressive agent (e.g., tocilizumab and/or mycophenolate mofetil), an anti-inflammatory agent (e.g., etanercept, or a non-steroidal anti-inflammatory agent (NSAID, such as aspirin and naproxen), an anticoagulant (e.g., heparin, aspirin or other known agent), and an antibiotic (e.g., ceftriaxone or other known agent).

In some embodiments, the method further includes administering a steroid, such as methylprednisolone. In some embodiments, the methylprednisolone is administered once per day or twice per day. The methylprednisolone may be administered at a dosage of about 2 mg/kg. The methylprednisolone may be administered prior to the transplant. The methylprednisolone may be administered one day prior to the transplant. The methylprednisolone may be administered on the same day as the transplant. The methylprednisolone may be administered after the transplant. The methylprednisolone may be administered one day, two days, three days, four days, five days, six days, one week, two weeks, three weeks, four weeks, two months, three months, four months, five months, and/or six months after the transplant. The methylprednisolone may be administered for a treatment period of 2 months, seven weeks, six weeks, five weeks, four weeks, three weeks, two weeks, one week, six days, five days, four days, three days, two days, or one day.

In some embodiments, the method further includes administering an antihistamine, such as diphenhydramine. The diphenhydramine may be administered at a dosage of about 50 mg. The diphenhydramine may be administered intravenously. The diphenhydramine may be administered prior to the transplant. The diphenhydramine may be administered one day prior to the transplant. The diphenhydramine may be administered on the same day as the transplant. The diphenhydramine may be administered after the transplant. The diphenhydramine may be administered one day, two days, three days, four days, five days, six days, one week, two weeks, three weeks, and/or four weeks after the transplant. The diphenhydramine may be administered for a treatment period of 2 months, seven weeks, six weeks, five weeks, four weeks, three weeks, two weeks, one week, six days, five days, four days, three days, two days, or one day.

In some embodiments, the method further includes administering an H2 receptor blocker, such as famotidine. The famotidine may be administered at a dosage of about 20 mg. The famotidine may be administered once per day or twice per day. The famotidine may be administered prior to the transplant.

The famotidine may be administered one day prior to the transplant. The famotidine may be administered on the same day as the transplant. The famotidine may be administered after the transplant. The famotidine may be administered one day, two days, three days, four days, five days, six days, one week, two weeks, three weeks, four weeks, five weeks, six weeks, seven weeks, and/or eight weeks after the transplant. The famotidine may be administered for a treatment period of 2 months, seven weeks, six weeks, five weeks, four weeks, three weeks, two weeks, one week, six days, five days, four days, three days, two days, or one day.

In some embodiments, the method further includes administering an antiviral agent, such as ganciclovir. The ganciclovir may be administered at a dosage of about 5 mg/kg. The ganciclovir may be administered prior to the transplant. The ganciclovir may be administered one day prior to the transplant.

The ganciclovir may be administered on the same day as the transplant. The ganciclovir may be administered after the transplant. The ganciclovir may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The ganciclovir may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The ganciclovir may be administered daily for a treatment period of at least one year, two years, three years, four years, five years, six years, seven years, eight years, nine years, ten years, or for the life of the subject.

In some embodiments, the method further includes administering a complement inhibitor, such as a Cl esterase inhibitor. The Cl esterase inhibitor may be administered at a dosage of about 20 U/kg.

The Cl esterase inhibitor may be administered prior to the transplant. The Cl esterase inhibitor may be administered one day prior to the transplant. The Cl esterase inhibitor may be administered on the same day as the transplant. The Cl esterase inhibitor may be administered after the transplant. The Cl esterase inhibitor may be administered one day, two days, three days, four days, five days, six days, and/or one week after the transplant.

In some embodiments, the method further includes administering an immunosuppressive agent, such as tocilizumab. The tocilizumab may be administered at a dosage of about 8 mg/kg. The tocilizumab may be administered intravenously. The tocilizumab may be administered on the same day as the transplant. The tocilizumab may be administered after the transplant. The tocilizumab may be administered one month, two months, three months, four months, five months, six months, seven months, eight months, nine months, ten months, eleven months, and/or twelve months after the transplant. The tocilizumab may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The tocilizumab may be administered monthly for a treatment period of at least one year, two years, three years, four years, five years, six years, seven years, eight years, nine years, ten years, or for the life of the subject.

In some embodiments, the method further includes administering an anti-inflammatory agent, such as etanercept. The etanercept may be administered at a dosage of about 0.7 IU/kg. The etanercept may be administered subcutaneously. The etanercept may be administered on the same day as the transplant. The etanercept may be administered after the transplant. The etanercept may be administered one week, two weeks, three weeks, one month, two months, three months, four months, five months, six months, seven months, eight months, nine months, ten months, eleven months, and/or twelve months after the transplant. The etanercept may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The etanercept may be administered weekly for a treatment period of at least one year, two years, three years, four years, five years, six years, seven years, eight years, nine years, ten years, or for the life of the subject. In some embodiments, the method further includes administering an immunosuppressive agent, such as mycophenolate mofetil. The mycophenolate mofetil may be administered at a dosage of about 20 mg/kg. The mycophenolate mofetil may be administered intravenously. The mycophenolate mofetil may be administered once per day or twice per day. The mycophenolate mofetil may be administered on the same day as the transplant. The mycophenolate mofetil may be administered after the transplant. The mycophenolate mofetil may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The mycophenolate mofetil may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The mycophenolate mofetil may be administered daily for a treatment period of at least one year, two years, three years, four years, five years, six years, seven years, eight years, nine years, ten years, or for the life of the subject.

In some embodiments, the method further includes administering an anticoagulant, such as heparin. The heparin may be administered on the same day as the transplant. The heparin may be administered after the transplant. The heparin may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The heparin may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The heparin may be administered daily for a treatment period of at least one year, two years, three years, four years, five years, six years, seven years, eight years, nine years, ten years, or for the life of the subject.

In some embodiments, the method further includes administering a non-steroidal anti-inflammatory agent (NSAID), such as aspirin. The aspirin may be administered at a dosage of about 81 mg. The aspirin may be administered after the transplant. The aspirin may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The aspirin may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The aspirin may be administered daily for a treatment period of at least one year, two years, three years, four years, five years, six years, seven years, eight years, nine years, ten years, or for the life of the subject.

In some embodiments, the method further includes administering an antibiotic, such as ceftriaxone. The ceftriaxone may be administered at a dosage of about 50 mg/kg. The ceftriaxone may be administered after the transplant. The ceftriaxone may be administered one day, two days, three days, four days, five days, six days, one week, and/or two weeks after the transplant. The ceftriaxone may be administered for a treatment period of one day, two days, three days, four days, five days, six days, one week, or two weeks. The ceftriaxone may be administered daily for a treatment period of one week.

A third aspect features a method of treating or reducing the risk of transplant rejection or increasing a duration of time before transplant rejection occurs in a human subject receiving or having received a porcine heart xenograft transplant. The method includes administering a humanized anti-CD40 antibody or antigen-binding fragment thereof that includes a heavy chain variable region including a CDRH1, CDRH2, and CDRH3 as set forth SEQ ID NOs: 1-3, respectively, and a light chain variable region including a CDRL1, CDRL2, and CDRL3 as set forth in SEQ ID NOs: 4-6, respectively. In some embodiments, the heavy chain variable region includes an amino acid sequence having at least 85% (e.g., at least 90%, 95%, 97%, 99%, or 100%) sequence identity to SEQ ID NO: 7, and the light chain variable region includes an amino acid sequence having at least 85% (e.g., at least 90%, 95%, 97%, 99%, or 100%) sequence identity to SEQ ID NO: 8. In some embodiments, the heavy chain variable region includes the amino acid sequence set forth in SEQ ID NO: 7, and the light chain variable region includes the amino acid sequence set forth in SEQ ID NO: 8. In some embodiments, the heavy chain includes the amino acid sequence set forth in SEQ ID NO: 9, and the light chain includes the amino acid sequence set forth in SEQ ID NO: 10. The method includes administering the anti-CD40 antibody or antigen-binding fragment thereof from about 1 hour to about 24 hours (e.g., from about 10 hours to about 24 hours) prior to the transplant and administering the anti-CD40 antibody or antigen-binding fragment thereof within about 1 hour to about 24 hours following the transplant, for example, once the subject achieves hemostasis. The antibody or antigen-binding fragment thereof may be administered subcutaneously or intravenously.

In some embodiments, the antibody or antigen-binding fragment thereof is administered within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant, for example, once the subject achieves hemostasis.

In some embodiments, the antibody or antigen-binding fragment thereof is administered one day, two days, three days, four days, five days, six days, one week, two weeks, three weeks, four weeks, two months, three months, four months, five months, six months, seven months, eight months, nine months, ten months, 11 months, and/or one year after the transplant.

In some embodiments, the antibody or antigen-binding fragment thereof is administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, about 1 mg/kg to about 20 mg/kg (e.g., about 5 mg/kg or about 10 mg/kg) of the antibody or antigen-binding fragment thereof is administered at least once every week, once every two weeks, once every three weeks, or once a month for a treatment period of one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, the antibody or antigen-binding fragment thereof is administered at least once every week, once every two weeks, once every three weeks, or once a month for a treatment period of one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, the method includes administering the antibody or antigen-binding fragment thereof as a an induction dose, e.g., as a divided dose that includes at least two doses: a first dose of about 1 mg/kg to about 20 mg/kg (e.g., a dose of about 10 mg/kg) that is administered prior to the transplant (e.g., from about 1 hour to about 24 hours, e.g., from about 10 hours to about 24 hours prior to the transplant (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant) and a second dose of about 1 mg/kg to about 20 mg/kg (e.g., a dosage of about 10 mg/kg) that is administered within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant (e.g., once the subject achieves hemostasis).

In certain embodiments, the induction dose administered prior to the transplant is a “conditioning dose”, which functions to prophylactically suppress the subject's immune system with an anti-CD40 antibody, or antigen-binding fragment thereof, prior to the transplant. Accordingly, the first induction dose can be administered as a divided dose that includes at least two doses: a conditioning dose of about 1 mg/kg to about 20 mg/kg (e.g., a dose of about 10 mg/kg) that is administered prior to the transplant (e.g., from about 1 hour to about 24 hours, e.g., from about 10 hours to about 24 hours prior to the transplant (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant) and an induction dose of about 1 mg/kg to about 20 mg/kg (e.g., a dosage of about 10 mg/kg) that is administered within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant (e.g., once the subject achieves hemostasis).

Alternatively, the two, divided induction doses administered before and after transplant can be administered as a single, undivided dose of about 1 mg/kg to about 20 mg/kg (e.g., either before the transplant or after the transplant (e.g., after the patient achieves hemostasis)). In one embodiment, a subject is administered a conditioning dose of about 1 mg/kg to about 20 mg/kg before the transplant (e.g., from about 1 hour to about 24 hours, e.g., from about 10 hours to about 24 hours prior to the transplant (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant).

The method may optionally include administering a second induction dose of the antibody or antigen-binding fragment thereof (e.g., as a divided dose with at least two separate doses administered 1-24 hours apart (e.g., 1-12 hours apart) or as an undivided dose) in an amount of from about 1 mg/kg to about 20 mg/kg (e.g., a dose of about 10 mg/kg) from about 1 day to about 10 days (e.g., about 1 day, 2 days, 3 days, 4 days, 5 days, 6 days, 7 days, 8 days, 9 days, or 10 days) following the transplant (e.g., after administration of a first induction dose). The method may optionally include administering a third induction dose (e.g., as a divided dose with at least two separate doses administered, e.g., 1-24 hours apart (e.g., 1-12 hours apart) or as an undivided dose) in an amount of from about 1 mg/kg to about 20 mg/kg (e.g., a dose of about 5 mg/kg or about 10 mg/kg). The second and/or third induction doses may be administered, e.g., at days 2, 3, 4, 5, 6, 7, 8, 9, and/or 10 following the transplant (e.g., the second induction dose following administration of the first induction dose and the third induction dose following administration of the second induction dose). Additional induction doses (e.g., fourth, fifth, etc., induction doses) may also be administered as a divided or undivided dose (e.g., in an amount of from about 1 mg/kg to about 20 mg/kg per dose (e.g., a dose of about 5 mg/kg or about 10 mg/kg)). When administered as an undivided dose, the second, third, fourth, and/or fifth induction doses, etc., may be administered in an amount of about 1 mg/kg to about 20 mg/kg per dose (e.g., a dose of about 5 mg/kg or about 10 mg/kg).

In some embodiments, the subject is administered one of more induction doses until the PK of the anti-CD40 antibody, or antigen-binding fragment thereof, in the subject's blood, plasma, or serum (e.g., peak and trough concentrations) is replicable or is predictable (e.g., at the desired therapeutic range). For example, in some embodiments, multiple induction doses are administered (as described herein) until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL).

In other embodiments, multiple induction doses (as described herein) are administered until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be about 20%, e.g., within 15%, 10%, 5% or less, of each other.

In another embodiment, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL)

In other embodiments, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be about 20%, e.g., within 15%, 10%, 5% or less, of each other. Optionally, the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week).

Once the PK of the antibody or antigen-binding fragment thereof in the subject's blood, plasma, or serum is replicable or predictable at a desired therapeutic range, the subject may transition from induction dosing to maintenance dosing. In certain embodiments, the subject transitions from induction dosing to maintenance dosing when (i) two or more C_{trough} of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other); and/or (ii) two or more Cₘₐₓ of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other).

Optionally, the C_{trough} and/or the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week). The method further includes, for example, treating the subject with a subsequent maintenance dose (e.g., after administration of the last induction dose) of the antibody or antigen-binding fragment thereof of about 1 mg/kg to about 20 mg/kg (e.g., a dose of about 5 mg/kg or about 10 mg/kg) that is administered once every week, once every two weeks, once every three weeks, or once a month (e.g., once every two or three weeks). The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject).

In some embodiments, the maintenance dose is provided to maintain a desired minimum blood, plasma, or serum concentration of the antibody or antigen-binding fragment thereof. For example, in some embodiments, it is desirable to maintain a minimum concentration (e.g., a trough concentration) within the range of about 20 μg/mL to about 200 μg/mL, such as a minimum trough concentration of at least 20 μg/mL (e.g., at least 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, or 150 μg/mL (e.g., at least 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL, e.g., from about 150 μg/mL to about 300 μg/mL)) of the antibody or antigen-binding fragment thereof in the blood, plasma, or serum. Accordingly, the method may include administering an induction dose or a maintenance does and monitoring the concentration of the antibody or antigen-binding fragment thereof in the blood, plasma, or serum. Once the concentration approaches the minimum threshold (e.g., about 150 μg/mL or about 20 μg/mL, or, e.g., within about 10%, 20%, 30%, 40%, or 50% of the minimum threshold), the method may include administering a maintenance dose (e.g., at a dosage of about 1 mg/kg to about 20 mg/kg, e.g., about 10 mg/kg) of the antibody or antigen-binding fragment thereof. These maintenance doses can be repeatedly administered following the transplant, e.g., once every week, once every two weeks, once every three weeks, or once a month (e.g., once every two or three weeks). The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject). The timing of administration of the maintenance dose and/or the timing between maintenance doses may be selected based on the PK profile of the antibody or antigen-binding fragment thereof in, e.g., the blood, plasma, or serum of the subject.

The subject may have been administered a therapeutic agent (e.g., an anti-CD20 antibody, such as rituximab) that depletes or reduces B cells in the subject. The subject may have been administered a therapeutic agent (e.g., anti-thymocyte globulin or anti-IL-2Rα receptor antibody) that depletes or reduces T cells in the subject.

In some embodiments, the method further includes administering one or more of a steroid (e.g., methylprednisolone), an antihistamine (e.g., diphenhydramine), an H2 receptor blocker (e.g., famotidine), an antiviral agent (e.g., ganciclovir), a complement inhibitor (e.g., a C1 esterase inhibitor), an immunosuppressive agent (e.g., tocilizumab and/or mycophenolate mofetil), an anti-inflammatory agent (e.g., etanercept, or a non-steroidal anti-inflammatory agent (NSAID, such as aspirin and naproxen), an anticoagulant (e.g., heparin and aspirin or other known agent), and an antibiotic (e.g., ceftriaxone or other known agent) to the subject before and/or after the transplant.

A fourth aspect features a method of treating or reducing the risk of transplant rejection or increasing a duration of time before transplant rejection occurs in a human subject receiving or having received a xenograft transplant. The method includes administering a humanized anti-CD40 antibody or antigen-binding fragment thereof having a heavy chain variable region with the amino acid sequence set forth in SEQ ID NO: 7, and a light chain variable region with the amino acid sequence set forth in SEQ ID NO: 8. The antibody or antigen-binding fragment thereof may be administered subcutaneously or intravenously, e.g., at a dosage of about 1 mg/kg to about 20 mg/kg (e.g., about 10 mg/kg), e.g., from about 10 hours to about 24 hours prior to the transplant. The method may further include administering the antibody or antigen-binding fragment thereof on the same day as the transplant. The method may further include administering the antibody or antigen-binding fragment thereof after the transplant. The antibody or antigen-binding fragment thereof may be administered within about 1 hour to about 24 hours after the subject reaches hemostasis following the transplant. The method may further include administering the antibody or antigen-binding fragment thereof once a week, once every other week, once every three weeks, or once a month after the transplant.

The method may further include intravenously administering an anti-CD20 antibody, such as rituximab, e.g., at a dosage of about 1 mg/kg to about 40 mg/kg, e.g., from about 10 hours to about 24 hours prior to the transplant. The anti-CD20 antibody (e.g., rituximab) may be administered after the transplant, e.g., three days, seven days, and/or fourteen days after the transplant. The method may further include intravenously administering anti-thymocyte globulin or anti-IL-2Rα receptor antibody at a dosage of about 1 mg/kg to about 10 mg/kg on the same day as the transplant.

In some embodiments, the method further includes administering one or more of a steroid (e.g., methylprednisolone), an antihistamine (e.g., diphenhydramine), an H2 receptor blocker (e.g., famotidine), an antiviral agent (e.g., ganciclovir), a complement inhibitor (e.g., a Cl esterase inhibitor), an immunosuppressive agent (e.g., tocilizumab and/or mycophenolate mofetil), an anti-inflammatory agent (e.g., etanercept, or a non-steroidal anti-inflammatory agent (NSAID, such as aspirin and naproxen), an anticoagulant (e.g., heparin and aspirin or other known agent), and an antibiotic (e.g., ceftriaxone or other known agent) to the subject before and/or after the transplant. In some embodiments, the xenograft transplant includes a cell, tissue, or organ, or portion thereof from a pig, cow, horse, dog, cat, sheep, goat, non-human primate (e.g., a macaque (e.g., a rhesus macaque or a cynomolgus macaque), a baboon, a marmoset, a monkey, and a chimpanzee), or gorilla. The xenograft transplant may be or includes a heart, kidney, lung, liver, pancreas, intestine, thymus, skin, eye, uterus, stem cell, bone, tendon, cornea, heart valve, nerve, vein, or a portion thereof. In some embodiments, the xenograft transplant includes a porcine heart. In some embodiments, the xenograft transplant has been genetically engineered to add, remove, or modify one or more xenoreactive antigens. In some embodiments, the xenograft transplant includes an organ or portion thereof from a host animal that has been genetically engineered to add, remove, or modify one or more xenoreactive antigens.

A fifth aspect features a method of treating or reducing a risk of transplant rejection or increasing a duration of time before transplant rejection occurs in a human subject receiving or having received a transplant (e.g., an allogeneic or xenogeneic transplant). The method includes a) administering intravenously or subcutaneously an induction dose of a humanized anti-CD40 antibody or antigen-binding fragment thereof as a divided dose, which includes a first dose of about 1 mg/kg to about 20 mg/kg from about 10 hours to about 24 hours prior to the transplant and a second dose of about 1 mg/kg to about 20 mg/kg, after the transplant once the subject achieves hemostasis, and b) administering subcutaneously or intravenously a maintenance dose of the humanized anti-CD40 antibody or antigen-binding fragment thereof at a dosage of about 1 mg/kg to about 20 mg/kg for treatment period of at least one month.

An induction dose (e.g., first induction dose) may, alternatively, be administered as a single undivided dose (e.g., of from about 1 mg/kg to about 20 mg/kg, e.g., about 10 mg/kg or 20 mg/kg)). Alternatively, an induction dose (e.g., first induction dose) may be administered as a divided dose of at least two doses (e.g., each dose of the divided dose contains from about 1 mg/kg to about 20 mg/kg, e.g., about 10 mg/kg), in which each dose is administered from about 1 hour to about 24 hours apart, e.g., 1 hour to 12 hours, apart.

In some embodiments, the subject is administered one of more additional induction doses (e.g., as a divided dose or an undivided dose, such as 1, 2, 3, 4, 5, 6, 7, 8, 9, or 10 additional induction doses), as described herein, until the PK of the anti-CD40 antibody or antigen-binding fragment thereof in the subject's blood, plasma, or serum (e.g., peak and trough concentrations) is replicable or predictable (e.g., in the desired therapeutic range). For example, in some embodiments, multiple induction doses are administered (as described herein) until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL).

In other embodiments, multiple induction doses (as described herein) are administered until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be about 20%, e.g., within 15%, 10%, 5% or less, of each other. In another embodiment, multiple induction doses (as described herein) are administered until the

PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL)

In other embodiments, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be about 20%, e.g., within 15%, 10%, 5% or less, of each other. Optionally, the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week).

Once the PK of the antibody or antigen-binding fragment thereof in the subject's blood, plasma, or serum is replicable or predictable at a desired therapeutic range, the subject may transition from induction dosing to maintenance dosing. In certain embodiments, the subject transitions from induction dosing to maintenance dosing when (i) two or more C_{trough} of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other); and/or (ii) two or more Cₘₐₓ of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other).

Optionally, the C_{trough} and/or the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week). The method further includes treating the subject with a maintenance dose of the antibody or antigen-binding fragment thereof (e.g., after the final induction dose) of about 1 mg/kg to about 20 mg/kg, or about 5 mg/kg to about 10mg/kg (e.g., a dose of about 5mg/kg or about 10 mg/kg) that is administered once every week, once every two weeks, once every three weeks, or once a month (e.g., once every two or three weeks). The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject).

In some embodiments, the maintenance dose is provided to maintain a minimum blood, plasma, or serum concentration of the humanized anti-CD40 antibody or antigen-binding fragment thereof of at least 150 μg/mL. In some embodiments, the maintenance dose is provided to maintain a minimum blood, plasma, or serum concentration of the humanized anti-CD40 antibody or antigen-binding fragment thereof within the range of at least about 20 μg/mL to about 200 μg/mL, such as at least 20 μg/ml.

In some embodiments, a blood, plasma, or serum concentration of the humanized anti-CD40 antibody or antigen-binding fragment thereof is used to guide redosing during maintenance dosing. For example, if a serum concentration drops below a desired or predetermined threshold or range (e.g., from about 20 μg/mL to about 300 μg/mL, e.g., from about 50 μg/mL to about 200 μg/mL, from about 50 μg/mL to about 150 μg/mL, or from about 80 μg/mL to about 150 μg/mL), the subject can be administered a maintenance dose. If the serum concentration drops, for example, more than 10%, e.g., more than 20%, 30%, 40%, or more) below a predetermined threshold or range, the subject can be administered one or more “reloading dose(s)” to increase the serum concentration to a desired therapeutic level (e.g., to a concentration of from about 20 μg/mL to about 300 μg/mL, e.g., from about 50 μg/mL to about 200 μg/mL, from about 50 μg/mL to about 150 μg/mL, or from about 80 μg/mL to about 150 μg/mL). In some embodiments, the maintenance dose may be administered at a greater dosage than a prior maintenance dose (e.g., a dose of greater than 10 mg/kg or 20 mg/kg) if it is administered following a drop below a desired minimum blood, plasma, or serum concentration (e.g., below about 150 μg/mL or below about 20 μg/mL). In some embodiments, one or more maintenance dose(s) may be administered at a shorter dosing interval than the prior maintenance doses if it is administered following a drop below a desired minimum blood, plasma, or serum concentration (e.g., below about 150 μg/mL or below about 20 μg/mL).

In certain embodiments, if during induction dosing or maintenance dosing, the subject has signs or symptoms of transplant rejection (e.g., elevated troponin levels or abnormal echocardiogram), the subject can be administered (i) one or more rescue dose(s) of an anti-CD40 antibody or antigen-binding fragment thereof; (ii) one or more increased dose(s) of the anti-CD40 antibody or antigen-binding fragment thereof relative to the dose level administered to the subject prior to the signs or symptoms of transplant rejection; or (iii) the anti-CD40 antibody or antigen-binding fragment thereof at an increased dose frequency relative to the dose frequency administered to the subject prior to relative to the dose level administered to the subject prior to the signs or symptoms of transplant rejection, or combinations of (i)-(iii). For example, a subject receiving a dose of about 10 mg/kg once per week may receive an increased dose of, for example, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg once per week. Alternatively, or in addition, the treatment frequency may be increased, such as from 10 mg/kg once per one or two weeks to 10 mg/kg twice per week, three times per week, four times per week, five times per week, six times per week, or seven times per week (e.g., once per day).

In some embodiments, the antibody or antigen-binding fragment thereof includes a heavy chain variable region with a CDRH1, CDRH2, and CDRH3 as set forth SEQ ID NOs: 1-3, respectively, and a light chain variable region with a CDRL1, CDRL2, and CDRL3 as set forth in SEQ ID NOs: 4-6, respectively.

In some embodiments, the heavy chain variable region includes an amino acid sequence having at least 85% (e.g., at least 90%, 95%, 97%, 99%, or 100%) sequence identity to SEQ ID NO: 7, and wherein the light chain variable region includes an amino acid sequence having at least 85% (e.g., at least 90%, 95%, 97%, 99%, or 100%) sequence identity to SEQ ID NO: 8. In some embodiments, the heavy chain variable region includes the amino acid sequence set forth in SEQ ID NO: 7, and the light chain variable region includes the amino acid sequence set forth in SEQ ID NO: 8. In some embodiments, the heavy chain includes the amino acid sequence set forth in SEQ ID NO: 9, and the light chain includes the amino acid sequence set forth in SEQ ID NO: 10.

In some embodiments, the method further includes including administering a first therapeutic agent that depletes or reduces B cells in the subject (e.g., anti-CD20 antibody, such as rituximab) and/or a second therapeutic agent that depletes or reduces T cells in the subject (e.g., anti-thymocyte globulin, e.g., THYMOGLOBULIN® or ATGAM®, or anti-IL-2Ra receptor antibody).

The first therapeutic agent may be administered prior to the transplant (e.g., from about 1 hour to about 24 hours, e .g., from about 10 hours to about 24, e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant). The first therapeutic agent may be administered after the transplant (e.g., one day, two days, three days, four days, five days, six days, one week, two weeks, three weeks, and/or four weeks after the transplant).

In some embodiments, the first therapeutic agent is administered for a treatment period of one day, two days, three days, four days, five days, six days, one week, two weeks, three weeks, or four weeks. In some embodiments, the first therapeutic agent is administered daily, weekly, or once every other week for a treatment period of one week, two weeks, three weeks, or four weeks.

In some embodiments, the first therapeutic agent (e.g., anti-CD20 antibody, such as rituximab) is administered at a dosage of about 1 mg/kg to about 40 mg/kg (e.g., about 10 mg/kg). In some embodiments, the first therapeutic agent is administered intravenously.

In some embodiments, the first therapeutic agent is rituximab. In some embodiments, the second therapeutic agent may be administered prior to the transplant (e.g., from about 10 hours to about 24 hours prior to the transplant, e.g., about 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant).

In some embodiments, the second therapeutic agent is administered on the same day as the transplant.

The second therapeutic agent may be administered after the transplant (e.g., one day, two days, three days, four days, five days, six days, one week, two weeks, three weeks, and/or four weeks after the transplant). In some embodiments, the second therapeutic agent is administered for a treatment period of one day, two days, three days, four days, five days, six days, one week, two weeks, three weeks, or four weeks. In some embodiments, the second therapeutic agent is administered daily, weekly, or once every other week for a treatment period of one week, two weeks, three weeks, or four weeks. In some embodiments, the second therapeutic agent is administered within about 1 hour to about 24 hours following the transplant and after the subject achieves hemostasis (e.g., once the subject achieves hemostasis).

In some embodiments, the second therapeutic agent (e.g., anti-thymocyte globulin, e.g., THYMOGLOBULIN® or ATGAM®, or anti-IL-2Ra receptor antibody) is administered at a dosage of about 1 mg/kg to about 10 mg/kg, e.g., about 2 mg/kg or about 5 mg/kg. In some embodiments, the second therapeutic agent is THYMOGLOBULIN® and is administered at a dosage of about 2 mg/kg. In some embodiments, the second therapeutic agent is ATGAM® and is administered at a dosage of about 5 mg/kg. The second therapeutic agent may be administered intravenously. In some embodiments, the second therapeutic agent is anti-thymocyte globulin.

A sixth aspect features a method of treating or reducing the risk of transplant rejection or increasing a duration of time before transplant rejection occurs in a human subject receiving or having received a porcine kidney xenograft transplant. The method includes administering a humanized anti-CD40 antibody or antigen-binding fragment thereof that includes a heavy chain variable region including a CDRH1, CDRH2, and CDRH3 as set forth SEQ ID NOs: 1-3, respectively, and a light chain variable region including a CDRL1, CDRL2, and CDRL3 as set forth in SEQ ID NOs: 4-6, respectively (e.g., as an induction dose (e.g., as a conditioning dose) or maintenance dose). In some embodiments, the heavy chain variable region includes an amino acid sequence having at least 85% (e.g., at least 90%, 95%, 97%, 99%, or 100%) sequence identity to SEQ ID NO: 7, and the light chain variable region includes an amino acid sequence having at least 85% (e.g., at least 90%, 95%, 97%, 99%, or 100%) sequence identity to SEQ ID NO: 8. In some embodiments, the heavy chain variable region includes the amino acid sequence set forth in SEQ ID NO: 7, and the light chain variable region includes the amino acid sequence set forth in SEQ ID NO: 8. In some embodiments, the heavy chain includes the amino acid sequence set forth in SEQ ID NO: 9, and the light chain includes the amino acid sequence set forth in SEQ ID NO: 10.

The method includes administering the anti-CD40 antibody or antigen-binding fragment thereof (e.g., as an induction dose that is a conditioning dose) from about 1 hour to about 24 hours (e.g., from about 10 hours to about 24 hours) prior to the transplant and administering the anti-CD40 antibody or antigen-binding fragment thereof within about 1 hour to about 24 hours following the transplant, for example, once the subject achieves hemostasis. The antibody or antigen-binding fragment thereof may be administered subcutaneously or intravenously. The induction dose of the anti-CD40 antibody or antigen-binding fragment thereof may be administered as a divided dose (e.g., of at least two doses, in which each dose is about 1 mg/kg to about 20 mg/kg (e.g., about 5 mg/kg or about 10 mg/kg)) or as an undivided dose (e.g., a dose of about 1 mg/kg to about 20 mg/kg (e.g., about 5 mg/kg or about 10 mg/kg)).

In some embodiments, the antibody or antigen-binding fragment thereof is administered within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant (e.g. as an induction dose), for example, once the subject achieves hemostasis.

In some embodiments, the antibody or antigen-binding fragment thereof is administered one day, two days, three days, four days, five days, six days, one week, two weeks, three weeks, four weeks, two months, three months, four months, five months, six months, seven months, eight months, nine months, ten months, 11 months, and/or one year after the transplant. In some embodiments, the antibody or antigen-binding fragment thereof is administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. In some embodiments, the antibody or antigen-binding fragment thereof is administered at least once every week, once every two weeks, once every three weeks, or once a month for a treatment period of one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, about 1 mg/kg to about 20 mg/kg (e.g., about 5 mg/kg or about 10 mg/kg) of the antibody or antigen-binding fragment thereof is administered at least once every week, once every two weeks, once every three weeks, or once a month for a treatment period of one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, the subject is administered one of more additional induction doses (e.g., as a divided dose or an undivided dose, such as 1, 2, 3, 4, 5, 6, 7, 8, 9, or 10 additional induction doses), as described herein, until the pharmacokinetics (PK) of the antibody or antigen-binding fragment thereof In some embodiments, the subject is administered one of more induction doses until the PK of the anti-CD40 antibody, or antigen-binding fragment thereof, in the subject's blood, plasma, or serum (e.g., peak and trough concentrations) is replicable or is predictable (e.g., in the desired therapeutic range). For example, in some embodiments, multiple induction doses are administered (as described herein) until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL).

In other embodiments, multiple induction doses (as described herein) are administered until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be about 20%, e.g., within 15%, 10%, 5% or less, of each other.

In another embodiment, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL)

In other embodiments, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be about 20%, e.g., within 15%, 10%, 5% or less, of each other. Optionally, the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week).

Once the PK of the antibody or antigen-binding fragment thereof in the subject's blood, plasma, or serum is replicable or predictable at a desired therapeutic range, the subject may transition from induction dosing to maintenance dosing. In certain embodiments, the subject transitions from induction dosing to maintenance dosing when (i) two or more C_{trough} of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other); and/or (ii) two or more Cₘₐₓ of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other).

Optionally, the C_{trough} and/or the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week). In some embodiments, a maintenance dose is provided to maintain a minimum blood, plasma, or serum concentration of the humanized anti-CD40 antibody or antigen-binding fragment thereof of at least 150 μg/mL. In some embodiments, the maintenance dose is provided to maintain a minimum blood, plasma, or serum concentration of the humanized anti-CD40 antibody or antigen-binding fragment thereof within the range of at least about 20 μg/mL to about 200 μg/mL, such as at least 20 μg/ml. In some embodiments, a serum concentration of the humanized anti-CD40 antibody or antigen-binding fragment thereof is used to guide redosing during maintenance dosing. For example, if a serum concentration drops below a desired or predetermined threshold or range (e.g., from about 20 μg/mL to about 300 μg/mL, e.g., from about 50 μg/mL to about 200 μg/mL, from about 50 μg/mL to about 150 μg/mL, or from about 80 μg/mL to about 150 μg/mL), the subject can be administered a maintenance dose. If the serum concentration drops, for example, more than 10%, e.g., more than 20%, 30%, 40%, or more) below a predetermined threshold or range, the subject can be administered one or more “reloading dose(s)” to increase the serum concentration to a desired therapeutic level (e.g., to a concentration of from about 20 μg/mL to about 300 μg/mL, e.g., from about 50 μg/mL to about 200 μg/mL, from about 50 μg/mL to about 150 μg/mL, or from about 80 μg/mL to about 150 μg/mL). In some embodiments, the maintenance dose may be administered at a greater dosage than a prior maintenance dose (e.g., a dose of greater than 10 mg/kg or 20 mg/kg) if it is administered following a drop below a desired minimum blood, plasma, or serum concentration (e.g., below about 150 μg/mL or below about 20 μg/mL). In some embodiments, one or more maintenance dose(s) may be administered at a shorter dosing interval than the prior maintenance doses if it is administered following a drop below a desired minimum blood, plasma, or serum concentration (e.g., below about 150 μg/mL or below about 20 μg/mL).

In certain embodiments, if during induction dosing or maintenance dosing the subject has signs or symptoms of transplant rejection (e.g., elevated troponin levels or abnormal echocardiogram), the subject can be administered (i) one or more rescue dose(s) of an anti-CD40 antibody or antigen-binding fragment thereof; (ii) one or more increased dose(s) of the anti-CD40 antibody or antigen-binding fragment thereof relative to the dose level administered to the subject prior to the signs or symptoms of transplant rejection; (iii) the anti-CD40 antibody or antigen-binding fragment thereof at an increased dose frequency relative to the dose frequency administered to the subject prior to relative to the dose level administered to the subject prior to the signs or symptoms of transplant rejection, or combinations of (i)-(iii). For example, a subject receiving a dose of about 10 mg/kg once per week may receive an increased dose of, for example, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg once per week. Alternatively, or in addition, the treatment frequency may be increased, such as from 10 mg/kg once per one or two weeks to 10 mg/kg twice per week, three times per week, four times per week, five times per week, six times per week, or seven times per week (e.g., once per day).

In any of the aspects discussed above, the method may include administering an antibody or antigen-binding fragment thereof that targets CD40 ligand (CD4OL, i.e., CD154) or an antagonist that targets CD154 in place of the anti-CD40 antibody or antigen-binding fragment thereof. Suitable antibodies or antigen-binding fragments thereof and antagonists that target CD154 include, for example, dapirolizumab, VIB4920, AT-1501, and SAR441344 (INX021).

In any of the aspects discussed above, the method may include monitoring and/or adjusting a dosage of the anti-CD40 antibody or antigen-binding fragment thereof in response to loss or dilution thereof as a result of a procedure during or following a transplant procedure, such as during peri- or post-operative care of the subject. In an embodiment, the subject may experience a reduction in the blood, plasma, or serum concentration of the anti-CD40 antibody or antigen-binding fragment thereof as a result of volume shifts due to a loss or infusion of blood or other fluids. In such an embodiment, the method may include redosing of the anti-CD40 antibody or antigen-binding fragment thereof to increase the blood, plasma, or serum concentration of the anti-CD40 antibody or antigen-binding fragment thereof in the subject. For example, the subject may be treated with an additional therapeutic intervention(s), such as dialysis, extracorporeal membrane oxygenation (ECMO), continuous veno-venous hemofiltration (CVVHW), renal replacement therapy (e.g., continuous hemofiltration and hemodialysis, intermittent hemodialysis and peritoneal dialysis) and the like, or infusion of liquids, e.g., albumin or crystalloid, that may reduce the level of the anti-CD40 antibody or antigen-binding fragment thereof in the blood, plasma, or serum of the subject or may dilute the blood, plasma, or serum concentration of the anti-CD40 antibody or antigen-binding fragment thereof. In another embodiment, the subject may experience a pleural effusion and may, for example, undergo a procedure, such as thoracentesis, to remove the fluid from the lungs. The anti-CD40 antibody or antigen-binding fragment thereof may be present in the pleural effusion, thus removing the anti-CD40 antibody or antigen-binding fragment from circulation. In other instances, the subject may require plasmapheresis, which removes plasma (and antibodies contained therein) that is separated from the blood of the subject. These therapeutic interventions, and/or the use of external instrumentation, may (directly or indirectly) reduce the blood, plasma, or serum cumulative amount or concentration of the anti-CD40 antibody or antigen-binding fragment thereof in the subject, which may require redosing or adjustment of the dose level or dosing frequency of the anti-CD40 antibody or antigen-binding fragment thereof, or of other therapeutic agents. The anti-CD40 antibody or antigen-binding fragment thereof may be re-administered to the subject, or the dosage and/or dosing frequency of the anti-CD40 antibody or antigen-binding fragment thereof may be increased, as needed, to restore or increase the blood, plasma, or serum concentration of the anti-CD40 antibody or antigen-binding fragment thereof in the subject to account for the loss of blood or other fluids, or dilution thereof, due to one or more of these procedures and/or the usage of the external instrumentation.

### Definitions

As used herein, the term “about” means +/−10% of the recited value.

As used herein, by “administering” is meant a method of giving a dosage of a pharmaceutical composition (e.g., an antibody or antigen-binding fragment thereof as described herein or any of the other agents described herein). The compositions utilized in the methods described herein can be administered, for example, intravenously and subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, by gavage, in cremes, or in lipid compositions, according to methods known in the art for the particular agent. The method of administration can vary depending on various factors (e.g., the components of the composition being administered and the severity of the condition being treated) and by using known guidance for the agent being administered.

The terms “antibody” and “immunoglobulin (Ig)” are used interchangeably in the broadest sense and include monoclonal antibodies (e.g., full-length or intact monoclonal antibodies) and polyclonal antibodies, and may also include certain antibody fragments. An antibody typically comprises both “light chains” and “heavy chains.” The light chains of antibodies (immunoglobulins) from any vertebrate species can be assigned to one of two clearly distinct types, called kappa (κ) and lambda (λ), based on the amino acid sequences of their constant domains.

By “hemostasis” is meant a status of a subject after a surgical procedure (e.g., following transplant surgery (e.g., after the thoracic cavity is sewn up by a surgeon) in which the flow of blood has substantially or completely ceased (e.g., no bleeding is apparent).

By “pharmaceutical composition” is meant any composition that contains a therapeutically or biologically active agent, such as an antibody or antigen-binding fragment thereof as described herein or any of the other agents described herein. For the purposes of this disclosure, pharmaceutical compositions include the active agent and one or more pharmaceutically acceptable carriers, excipients, or diluents known in the art to be suitable for delivering a therapeutic or biologically active agent.

By “sequence identity” or “sequence similarity” is meant that the identity or similarity, respectively, between two or more amino acid sequences, or two or more nucleotide sequences, is expressed in terms of the identity or similarity between the sequences. Sequence identity can be measured in terms of “percentage (%) identity,” in which a higher percentage indicates greater identity shared between the sequences. Sequence similarity can be measured in terms of percentage similarity (which takes into account conservative amino acid substitutions); the higher the percentage, the more similarity shared between the sequences. Homologs or orthologs of nucleic acid or amino acid sequences possess a relatively high degree of sequence identity/similarity when aligned using standard methods. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications. Sequence identity/similarity can be determined across all or a defined portion of the two or more sequences compared.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a KD of less than 100 nM. For example, an antibody or antigen-binding fragment thereof that specifically binds to an antigen will bind to the antigen with a KD of up to 100 nM (e.g., between 1 pM and 100 nM). An antibody or antigen-binding fragment thereof that does not exhibit specific binding to a particular antigen or epitope thereof will exhibit a KD of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A

Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

A “subject” is a vertebrate, such as a mammal (e.g., a primate and a human, in particular a human with underlying health conditions (e.g., cardiovascular disease) that necessitates transplantation of a cell, tissue, or organ). A subject to be treated according to the methods described herein (e.g., a human, is one in need of a cell, tissue, or organ transplant. Diagnosis may be performed by any suitable means. One skilled in the art will understand that a subject to be treated according to the disclosure may have been subjected to standard tests or may have been identified, without examination, as one with suspected tissue or organ failure or one in need of restoration of cellular, tissue, or organ function. The methods of treating a human subject with a composition described herein (e.g., KPL-404) are particularly useful in treating, reducing the risk of, and/or mitigating or inhibiting cell, tissue, or organ transplant rejection.

As used herein, and as well understood in the art, “treatment” is an approach for obtaining beneficial or desired results, such as clinical results. Beneficial or desired results can include, but are not limited to, alleviation or amelioration of one or more symptoms of, or the occurrence of, transplant rejection (e.g., rejection of a cell, tissue, or organ transplant); diminishment of the extent of, or a extension of a duration time before the occurrence of, transplant rejection; stabilization (e.g., not worsening) of a transplanted cell, tissue, or organ; or a delay or slowing of the progress of rejection of a cell, tissue, or organ transplant, whether detectable or undetectable. A treatment can include one or more therapeutic agents, such as one or more of the compositions described herein and/or one or more additional therapeutic agents. Additional therapeutic agents can include agents that inhibit the immune response (e.g., an anti-inflammatory agent, a steroid, an H2 receptor blocker, etc.). A treatment can include one or more therapeutic interventions, such as surgery.

### BRIEF DESCRIPTION OF THE DRAWINGS

FIG. 1 is a set of graphs showing flow cytometric analysis of peripheral blood monocytes (PBMCs) performed on days −1, 0, 1, and 3 relative to a xenograft heart transplant. The following dosages indicate the cumulative dose of the respective drugs administered to the subject at the indicated time of blood sample collection for flow cytometry. Day −1: Rituximab 375 mg/m²; Day 0: status post (s/p): Rituximab 375mg/m²; Day 1: s/p: Rituximab 375mg/m² and s/p 1mg/kg ATG; Day 3: s/p Rituximab 375mg/m², s/p 2 mg/kg ATG.

FIG. 2 is a graph showing flow cytometric analysis of lymph nodes performed prior to chest closure following a xenograft heart transplant (day 2). At time of chest closure: s/p cumulative dose administered: Rituximab 375mg/m², s/p 2 mg/kg ATG.

FIG. 3 is a graph showing troponin levels (ng/mL) per day following xenograft heart transplant. FIG. 4 is a graph showing blood serum concentration of KPL-404 as a function of time. The top curve provides data from a Phase 1 study in normal healthy volunteers. The bottom curve provides data in a human subject following xenograft heart transplant.

FIG. 5 is a graph showing a time course of KPL-404 serum concentrations as a function of time relative to the heart transplant (horizonal line corresponding to xenotransplantation). Also shown as horizontal lines are time courses of abdominal surgery, continuous veno-venous hemofiltration (CVVHW) (hemofiltration), and extracorporeal membrane oxygenation (ECMO).

FIGS. 6A-6F are graphs showing B cell and T cell counts in peripheral blood following the heart transplant in the baboon. FIG. 6A shows percentage of T and B cells in peripheral blood relative to total blood lymphocyte count. FIG. 6B shows percentages of CD4 and CD8 T cells in peripheral blood relative to total blood CD4 and CD8 T cell count. FIG. 6C shows percentage of Treg cells in peripheral blood relative to total blood T reg cell count. FIG. 6D shows absolute numbers of T cells and B cells in peripheral blood. FIG. 6E shows absolute numbers of CD4 and CD8 T cells in peripheral blood. FIG. 6F shows absolute number of Treg cells in peripheral blood. These graphs indicate that T cells were still present in the animal at the time of transplant and that T cell count steadily increased over time soon after transplant, while B cell counts remained low after transplant.

FIG. 7 is a graph showing an extended time course (relative to FIG. 5) of KPL-404 serum concentrations as a function of time relative to the heart transplant (horizonal line corresponding to xenotransplantation). Also shown as horizontal lines are time courses of abdominal surgery, continuous veno-venous hemofiltration (CVVHW) (hemofiltration), and extracorporeal membrane oxygenation (ECMO).

FIGS. 8A and 8B are graphs showing B cell and T cell counts in peripheral blood following the heart transplant in the human subject. FIG. 8A shows percentage of T cell (CD3+) and B cells (CD20+) in peripheral blood relative to total blood lymphocyte count. FIG. 8B shows percentages of CD4+ and CD8+ T cells in peripheral blood relative to total blood CD4+ and CD8+ T cell count.

FIG. 9 show clinical details during postoperative course, labeled in the upper x-axis in relation to donor specific endothelial IgM, IgG and Troponin I levels.

### DETAILED DESCRIPTION

The disclosure features methods of treating or reducing the risk of transplant rejection or increasing a duration of time before transplant rejection occurs in a human subject. The subject may have received or is receiving a transplant, such as a cell, organ or tissue transplant. The transplant may be an allogeneic transplant or a xenograft transplant. In general, the methods include administration of an antagonist that targets CD40 or CD154. For example, the methods may include administration of an anti-CD40 antibody or antigen binding fragment thereof, e.g., prior to, on the day of, and/or following the transplant. The antagonists may be anti-CD40 antibodies or antigen-binding fragments thereof or the antagonists may be, e.g., antibodies or antigen-binding fragments thereof that bind to CD154. The antagonists can block the ability of CD40 to bind CD154 and do so without activating the cell expressing CD40 (e.g., a B cell), thus suppressing the immune system to treat or inhibit transplant rejection. The methods described herein may further include administration of an agent that depletes or reduces B cells in the subject (e.g., rituximab) and/or an agent that depletes or reduces T cells in the subject (e.g., anti-thymocyte globulin). The antibody or antigen-binding fragment thereof can be one derived from the murine 2C10 antibody and humanized variants thereof, e.g., as described in PCT Pub. Nos. WO 2012/125569 and WO 2017/040932, which are herein incorporated by reference in their entirety. The methods and the pharmaceutical compositions used therein are described in more detail below.

Antibodies and Antigen-Binding Fragments Thereof and Antagonists that target CD40 or CD154

The methods described herein include administration of an antagonist (e.g., an antibody or antigen-binding fragment thereof) that disrupts the CD40-CD154 interaction. The antagonist may target CD40 or may target CD154. In some embodiments, the methods described herein include administration of an antibody or antigen-binding fragment thereof, e.g., derived from the murine 2C10 antibody. The heavy chain variable regions, light chain variable regions, and CDRs of certain anti-CD40 antibodies described herein are shown in Table 1.

TABLE 1
KPL-404 antibody sequences

| | Chain, | | SEQ ID |
|---|---|---|---|
| Name | Region | Sequence | NO: |
| KPL-404 | Heavy chain, | YTFTNYWMH | 1 |
| | CDR1 | | |
| KPL-404 | Heavy chain, | YINPSNDYTKYNQKFKD | 2 |
| | CDR2 | | |
| KPL-404 | Heavy chain, | QGFPY | 3 |
| | CDR3 | | |
| KPL-404 | Light chain, | SASSSVSYMH | 4 |
| | CDR1 | | |
| KPL-404 | Light chain, | DTSKLAS | 5 |
| | CDR2 | | |
| KPL-404 | Light chain, | HQLSSDPFT | 6 |
| | CDR3 | | |
| KPL-404 | Heavy chain, | QVQLVQSGAEVKKPGASVKVSCKASGYTFTNYWMHW | 7 |
| | variable | VRQAPGQRLEWIGYINPSNDYTKYNQKFKDRATLTA | |
| | region | DKSANTAYMELSSLRSEDTAVYYCARQGFPYWGQGT | |
| | | LVTVSS | |
| KPL-404 | Light chain, | EIVLTQSPATLSLSPGERATLSCSASSSVSYMHWYQ | 8 |
| | variable | QKPGQAPRRWIYDTSKLASGVPARFSGSGSGTDYTL | |
| | region | TISSLEPEDFAVYYCHQLSSDPFTFGGGTKVEIK | |
| KPL-404 | Heavy chain | QVQLVQSGAEVKKPGASVKVSCKASGYTFTNYWMHW | 9 |
| | (with IgG4 | VRQAPGQRLEWIGYINPSNDYTKYNQKFKDRATLTA | |
| | constant | DKSANTAYMELSSLRSEDTAVYYCARQGFPYWGQGT | |
| | domain) | LVTVSSASTKGPSVFPLAPCSRSTSESTAALGCLVK | |
| | | DYFPEPVTVSWNSGALTSGVHTFPAVLQSSGLYSLS | |
| | | SVVTVPSSSLGTKTYTCNVDHKPSNTKVDKRVESKY | |
| | | GPPCPPCPAPEFLGGPSVFLFPPKPKDTLMISRTPE | |
| | | VTCVVVDVSQEDPEVQFNWYVDGVEVHNAKTKPREE | |
| | | QFNSTYRVVSVLTVLHQDWLNGKEYKCKVSNKGLPS | |
| | | SIEKTISKAKGQPREPQVYTLPPSQEEMTKNQVSLT | |
| | | CLVKGFYPSDIAVEWESNGQPENNYKTTPPVLDSDG | |
| | | SFFLYSRLTVDKSRWQEGNVFSCSVMHEALHNHYTQ | |
| | | KSLSLSPGK | |
| KPL-404 | Light chain | EIVLTQSPATLSLSPGERATLSCSASSSVSYMHWYQ | 10 |
| | (with kappa | QKPGQAPRRWIYDTSKLASGVPARFSGSGSGTDYTL | |
| | constant | TISSLEPEDFAVYYCHQLSSDPFTFGGGTKVEIKRT | |
| | domain) | VAAPSVFIFPPSDEQLKSGTASVVCLLNNFYPREAK | |
| | | VQWKVDNALQSGNSQESVTEQDSKDSTYSLSSTLTL | |
| | | SKADYEKHKVYACEVTHQGLSSPVTKSFNRGEC | |

In certain embodiments, the antibody or antigen-binding fragment thereof includes (e.g., consists of) a heavy chain variable region including an amino acid sequence having at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the heavy chain variable region amino acid sequence as set forth in SEQ ID NO: 7.

In certain embodiments, the antibody or antigen-binding fragment thereof includes (e.g., consists of) a light chain variable region including an amino acid sequence having at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to a light chain variable region amino acid sequence as set forth in SEQ ID NO: 8.

In certain embodiments, the antibodies or antigen-binding fragments thereof include (e.g., consists of) both a heavy chain variable region including an amino acid sequence having at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to a heavy chain variable region amino acid sequence as set forth in SEQ ID NO: 7, and a light chain variable region including an amino acid sequence having at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to a variable light chain amino acid sequence as set forth in SEQ ID NO: 8.

In certain embodiments, the antibodies or antigen-binding fragments thereof include (e.g., consists of) a heavy chain region including an amino acid sequence having at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to a heavy chain with the amino acid sequence as set forth in SEQ ID NO: 9, and a light chain region including an amino acid sequence having at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the light chain amino acid sequence as set forth in SEQ ID NO: 10.

In certain embodiments, a heavy chain variable region of the antibody or antigen-binding fragment thereof includes complementarity determining regions (CDRs) that are at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the CDRs of a heavy chain variable region of the KPL-404 antibody (CDR1, CDR2 and CDR3 as set forth in SEQ ID NOs: 1, 2, 3, respectively).

In certain embodiments, the light chain variable region of the antibody or antigen-binding fragment thereof includes CDRs that are at least about 70%, at least about 75%, at least about 80%, at least about 85%, at least about 90%, at least about 95%, at least about 99%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to the CDRs of a light chain variable region of the KPL-404 antibody (CDR1, CDR2 and CDR3 as set forth in SEQ ID NOs: 4, 5, 6, respectively).

In certain embodiments, the heavy chain includes the CDRs as set forth in SEQ ID NOs: 1-3, respectively, and the light chain includes the CDRs as set forth in SEQ ID NOs: 4-6, respectively.

In certain embodiments, the heavy chain has at least 80% (e.g., at least 85%, 90%, 95%, 97%, 99%, or 100%) sequence identity to SEQ ID NO: 7 and the CDRs set forth in SEQ ID NOs: 1-3, respectively, and the light chain has at least 80% (e.g., at least 85%, 90%, 95%, 97%, 99%, or 100%) sequence identity to SEQ ID NO: 8 and the CDRs set forth in SEQ ID NOs: 4-6, respectively. Other exemplary anti-CD40 antibodies or antibody fragments thereof are, e.g., BI 55064, which is described in U.S. Pat. No. 8,591,900, iscalimab, which is described in U.S. Pat. No. 8,828,396, bleselumab, which is described in U.S. Pat. No. 9,125,893 and PCT Pub. No. WO2017100305, BMS-986325, which is described in US Pub. No. US20210054090 and PCT Pub. No. WO2021236546, each of the foregoing are hereby incorporated by reference in their entirety, including the amino acid and nucleic acid sequences described therein.

In some aspects, an antibody or antigen-binding fragment thereof that targets CD40 ligand (CD40L, i.e., CD154) may be contemplated within the scope of the disclosure. Also contemplated are antagonists that target CD154. Suitable antibodies or antigen-binding fragments thereof and antagonists that target CD154 include, for example, dapirolizumab, which is described in U.S. Pat. No. 8,293,237; VIB4920, which is described in U.S. Pat. No. 10,000,553; AT-1501, which is described in U.S. Pat. No. 10,106,618; and SAR441344 (INX021), which is described in US Pub. No. US20210145966, each of the foregoing are hereby incorporated by reference in their entirety, including the amino acid and nucleic acid sequences described therein.Also within the scope of the disclosure are antibodies or antigen-binding fragments thereof in which specific amino acids have been substituted, deleted, or added. These alternations do not have a substantial effect on the peptide's biological properties such as binding activity. For example, antibodies may have amino acid substitutions in the framework region, such as to improve binding to the antigen. In another example, a selected, small number of acceptor framework residues can be replaced by the corresponding donor amino acids. The donor framework can be a mature or germline human antibody framework sequence or a consensus sequence. Guidance concerning how to make phenotypically silent amino acid substitutions is provided in, e.g., Bowie et al. (Science, 247: 1306-1310, 1990), Cunningham et al. (Science, 244: 1081-1085, 1989), Ausubel (ed.) (Current Protocols in Molecular Biology, John Wiley and Sons, Inc., 1994), T. Maniatis, E. F. Fritsch and J. Sambrook (Molecular Cloning: A Laboratory Manual, Cold Spring Harbor laboratory, Cold Spring Harbor, N.Y., 1989), Pearson (Methods Mol. Biol. 243:307-31, 1994), and Gonnet et al. (Science 256:1443-45, 1992); each of which is incorporated herein by reference.

The polypeptides described herein may be a functionally active variant of the antibodies or antigen-binding fragments thereof disclosed herein, e.g., with less than about 30%, about 25%, about 20%, about 15%, about 10%, about 5% or about 1% amino acid residues substituted or deleted but that retain essentially the same immunological properties including, but not limited to, binding to CD40.

In some embodiments, the dissociation constant (KD) of the antibody or antigen-binding fragment thereof is less than about 1×10⁻⁸ M, e.g., less than about 1×10⁻⁹ M. The antibodies or antigen-binding fragments thereof may also include one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art.

### Methods

The methods described herein include administration of a pharmaceutical composition containing an antibody or antigen-binding fragment (e.g., an antibody or antigen-binding fragment thereof can block the ability of CD40 to bind CD154) as described herein to a subject receiving or having received a transplant, such as a cell, organ, or tissue transplant (e.g., a xenotransplant). The methods may treat or inhibit transplant rejection in the subject or increase a duration of time before transplant rejection occurs. For example, the methods may increase the duration of time (e.g., by at least 10%, 20%, 30%, 40%, 50%, 60%, 70%, 80%, 90%, 100%, or more). The methods may inhibit transplant rejection for at least one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

The methods described herein may include administering to a subject an induction therapy during which the subject is administered one or more doses (e.g., induction doses) of an anti-CD40 antibody or antigen-binding fragment thereof in the perioperative period to cover the immediate post-transplant phase as the period with the highest risk of hyper acute and acute rejection of the transplant. In some embodiments, the induction therapy includes a fixed dosing regimen, wherein the subject is administered an anti-CD40 antibody or antigen-binding fragment thereof at a fixed or weight-based dose level, and at a fixed or predetermined dosing interval. In other embodiments, the induction therapy includes a pharmacokinetics (PK) target-based dosing regimen, wherein the subject is administered an anti-CD40 antibody or antigen-binding fragment thereof to achieve a desired blood, plasma, or serum concentration (e.g., peak and/or trough concentration) of the anti-CD40 antibody or antigen-binding fragment thereof sufficient to inhibit or suppress CD40 activity in the subject. In some embodiments, the induction therapy continues until the anti-CD40 antibody or antigen-binding fragment thereof achieves replicable or predictable PK in the subject.

A PK that is “replicable” means that when, at a given dose and dose interval, there is dose-to-dose consistency, with C_{trough} being observed at or near a desired range (e.g., a therapeutic range) and with Cₘₐₓ being observed at or near a desired range, in each case, over two or more consecutive administrations of the antibody or antigen-binding fragment thereof. Once the anti-CD40 antibody or antigen-binding fragment thereof has a replicable or predictable PK in the subject, a maintenance therapy is administered to the subject.

Method of determining whether PK is predictable are known in the art. Pharmacokineticists routinely employ methods of generating a PK analysis to predict a drug's PK behavior in a subject, such as non-compartmental analysis (NCA), which is a standard, efficient, and effective method for estimating PK parameters and to develop a model to describe the PK of a drug in a subject. In performing the PK analysis, a pharmacokineticist identifies data that are insufficient to generate a PK model and should therefore be excluded. Reasons for excluding data include, for example, an anomalous concentration value, lack of exposure when exposure was expected, and dose failure.

The method may include administering an anti-CD40 antibody or antigen-binding fragment thereof from about 1 hour to about 24 hours, e.g., from about 10 hours to about 24 hours, prior to the transplant and administering the anti-CD40 antibody or antigen-binding fragment thereof within about 1 hour to about 24 hours following the transplant, for example, once the subject achieves hemostasis. The antibody or antigen-binding fragment thereof may be administered subcutaneously or intravenously, e.g., at a dosage of about 1 mg/kg to about 20 mg/kg, e.g., about 5 mg/kg or about 10 mg/kg.

The antibody or antigen-binding fragment thereof may be administered prior to the transplant, e.g., from about 1 hour to about 24 hours, e.g., from about 10 hours to about 24 hours prior to the transplant, e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant. The antibody or antigen-binding fragment thereof may be administered on the same day as the transplant. The antibody or antigen-binding fragment thereof may be administered after the transplant. For example, the antibody or antigen-binding fragment thereof may be administered following the transplant within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant and, for example, once the subject achieves hemostasis. In a certain embodiment, the antibody or antigen-binding fragment thereof is administered after the transplant once the subject achieves hemostasis.

In some embodiments, the antibody or antigen-binding fragment thereof is administered one day, two days, three days, four days, five days, six days, one week, two weeks, three weeks, four weeks, two months, three months, four months, five months, six months, seven months, eight months, nine months, ten months, 11 months, and/or one year after the transplant. In some embodiments, the antibody or antigen-binding fragment thereof is administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

In some embodiments, the antibody or antigen-binding fragment thereof is administered at least once per week, once every two weeks, once every three weeks, or once a month for a treatment period of one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject.

The methods described herein may include administering the antibody or antigen-binding fragment thereof as a an induction dose that includes two doses: a first dose of about 1 mg/kg to about 20 mg/kg (e.g., a dose of about 5 mg/kg or about 10 mg/kg) that is administered prior to the transplant (e.g., from about 1 hour to about 24 hours, e.g., from about 10 hours to about 24 hours prior to the transplant (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant) and a second dose of about 1 mg/kg to about 20 mg/kg (e.g., a dosage of about 5 mg/kg or about 10 mg/kg) that is administered within about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours) following the transplant (e.g., once the subject achieves hemostasis). A dose administered to the subject prior to the transplant is also referred to herein as a “conditioning dose”. The conditioning dose is a part of the induction therapy that functions to prophylactically suppress the subject's immune system with an anti-CD40 antibody, or antigen-binding fragment thereof, prior to the transplant.

Alternatively, an induction dose may be administered as a single undivided dose of the antibody or antigen-binding fragment thereof (e.g., a dose of about 1 mg/kg to about 20 mg/kg (e.g., a dose of about 5 mg/kg or about 10 mg/kg)). As is described above, the induction dose may also be administered as a divided dose that includes at least two doses of the anti-CD40 antibody or antigen-binding fragment thereof. Whether an induction dose is administered as a single, undivided dose or a divided dose will depend on the dosing restrictions of the anti-CD40 antibody or antigen-binding fragment thereof, e.g., limits set by the drug's prescribing information, or as otherwise imposed by relevant clinical experience or restrictions imposed by applicable regulatory authorities. For example, where a target blood, plasma, or serum concentration of the anti-CD40 antibody or antigen-binding fragment thereof is desired, but the dose level required to achieve this target concentration as a single dose is not recommended due to limits set by the drug's prescribing information or a lack of clinical experience administering the single dose, the drug can be administered as a divided dose to achieve the target concentration. In certain embodiments, an induction dose that is a divided dose may be administered as a first dose and a second dose of the antibody or antigen-binding fragment thereof that are administered about 1 hour to about 24 hours apart, e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours apart, such as from about 1 to about 12 hours apart). For example, the first divided dose of the induction dose may involve administration of from about 1 mg/kg to about 20 mg/kg of the antibody or antigen-binding fragment thereof (e.g., a dosage of about 10 mg/kg) and a second divided dose of the induction dose may involve administration of from about 1 mg/kg to about 20 mg/kg of the antibody or antigen-binding fragment thereof (e.g., a dosage of about 10 mg/kg). The second divided dose of the induction dose may be administered about 1 hour to about 24 hours (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours, such as from about 1 to about 12 hours) after the subject receives the first divided dose of the induction dose. For example, the method may involve administration of a divided dose of an induction dose of 20 mg/kg of the antibody or antigen-binding fragment thereof, in which the first divided dose provides about 10 mg/kg of the antibody or antigen-binding fragment thereof and the second divided dose provides about 10 mg/kg of the antibody or antigen-binding fragment thereof.

The method may include administration of multiple induction doses (e.g., either as undivided doses or as divided doses). As noted above, induction therapy is administered during the perioperative period to cover the immediate post-transplant phase as the period with the highest risk of hyperacute or acute rejection of the transplant and, in accordance with the invention, is carried out before the anti-CD40 antibody or antigen-binding fragment thereof has attained a replicable or predictable PK profile. Without wishing to be bound by any particular theory, beginning on the day of the transplant and days, weeks or even months thereafter, the drug may not attain a replicable or predictable PK profile in the subject as a result of, for example, target-mediated drug disposition (TMDD) in the subject receiving a transplant. Perioperative care can also contribute to a variable and unpredictable PK profile of the drug in the subject, including, without limitation, fluid loss as a result of surgical intervention (both during the transplant procedure and any required post-transplant surgery or procedure), medical devices that circulate the subject's bodily fluids and affect the drug's PK in the subject (e.g., extracorporeal membrane oxygenation, continuous veno-venous hemofiltration, dialysis, therapeutic plasma exchange, plasmapheresis, hemodilution, and the like), for example, by the drug non-specifically binding to tubing and/or filters in the device(s), and/or by increasing the volume of the subject's bodily fluids (e.g., by administering crystalloid or albumin thereby reducing the blood, serum or plasma concentration of the drug). Thoracentesis may also be used to reduce pleural effusion, which could result in a reduction in the blood, serum, or plasma concentration of the anti-CD40 antibody or antigen-binding fragment thereof via loss in the pleural effusion.

For example, the method may include administering 1, 2, 3, 4, 5, 6, 7, 8, 9, 10, or more induction doses (e.g., from about 1 to about 5 induction doses, such as, e.g., about 3 or about 4 induction doses). In some embodiments, each induction dose (e.g., first induction dose, second induction dose, third induction dose, and/or fourth induction dose, or more) is a divided dose that includes first and second doses of the antibody or antigen-binding fragment thereof (e.g., in an amount of about 1 mg/kg to about 20 mg/kg per dose) that are, e.g., administered between 1 and 24 hours apart (e.g., 1 to 12 hours apart)). Alternatively, the first induction dose is a divided dose and any additional induction dose(s) (e.g., first, second, third, and/or fourth, or more induction dose(s)) is/are administered as either a divided dose of the antibody or antigen-binding fragment thereof (e.g., each induction dose including at least two doses in an amount of about 1 mg/kg to about 20 mg/kg (e.g., 5 mg/kg or 10 mg/kg) of the antibody or antigen-binding fragment thereof per dose) or an undivided dose of the antibody or antigen-binding fragment thereof (e.g., each induction dose being a single dose in an amount of about 1 mg/kg to about 20 mg/kg (e.g., 5 mg/kg or 10 mg/kg) of the antibody or antigen-binding fragment thereof). In another embodiment, the first induction dose is a divided dose and any additional induction dose(s) (e.g., first, second, third, and/or fourth, or more induction dose(s)) is/are administered as a divided dose (e.g., each induction dose including at least two doses in an amount of about 1 mg/kg to about 20 mg/kg of the antibody or antigen-binding fragment thereof per dose). In another embodiment, the first induction dose is a divided dose and any additional induction dose(s) (e.g., first, second, third, and/or fourth, or more induction dose(s)) is/are administered as an undivided dose (e.g., each induction dose being a single dose in an amount of about 1 mg/kg to about 20 mg/kg (e.g., 5 mg/kg or 10 mg/kg) of the antibody or antigen-binding fragment thereof). In some embodiments, a second induction dose is administered from about 1 day to about 14 days (e.g., 1 day, 2 days, 3 days, 4 days, 5 days, 6 days, 7 days, 8 days, 9 days, 10 days, 11 days, 12 days, 13 days, or 14 days) following administration of the first induction dose. In other embodiments, second, third, fourth, and/or fifth induction doses are administered from about 1 day to about 14 days apart (e.g., 1 day, 2 days, 3 days, 4 days, 5 days, 6 days, 7 days, 8 days, 9 days, 10 days, 11 days, 12 days, 13 days, or 14 days apart).

In some embodiments, the subject is administered one of more induction doses until the pharmacokinetics (PK) of the anti-CD40 antibody or antigen-binding fragment thereof in the subject's blood, plasma, or serum (e.g., peak and trough concentrations) is replicable or predictable (e.g., the desired therapeutic range). For example, in some embodiments, multiple induction doses are administered (as described herein) until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL).

In other embodiments, multiple induction doses (as described herein) are administered until the PK of the anti-CD40 antibody, or antigen-binding fragment thereof, in the subject's blood, plasma, or serum is replicable or is predictable, wherein two or more C_{trough} of the antibody or antigen-binding fragment thereof in the subject's blood, plasma, or serum determined after two or more administrations are, or can predicted to be about 20%, e.g., within 15%, 10%, 5% or less, of each other.

In another embodiment, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be, the same or in a similar range of about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL,160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, or 300 ug/mL)

In other embodiments, multiple induction doses (as described herein) are administered until the PK of the anti-CD40 antibody, or antigen-binding fragment thereof, in the subject's blood, plasma, or serum is replicable or is predictable, wherein two or more Cₘₐₓ of the antibody or antigen-binding fragment thereof in the subject's blood, plasma, or serum determined after two or more administrations are, or can predicted to be about 20%, e.g., within 15%, 10%, 5% or less, of each other. Optionally, the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week).

Once the PK of the antibody or antigen-binding fragment thereof in the subject's blood, plasma, or serum is replicable or predictable at a desired therapeutic range, the subject may transition from induction dosing to maintenance dosing. In certain embodiments, the subject transitions from induction dosing to maintenance dosing when (i) two or more C_{trough} of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other); and/or (ii) two or more Cₘₐₓ of the antibody or antigen-binding fragment thereof in the subject's blood, plasma, or serum determined after two or more administrations are, or can predicted to be, the same or in a similar range (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL; or within about 20%, e.g., within about 15%, 10%, 5% or less, of each other).

Optionally, the C_{trough} and/or the Cₘₐₓ of the antibody or antigen-binding fragment thereof in blood, plasma, or serum may be determined over a period of, e.g., 1 to 7 days, 1 to 14 days, 1 to 21 days, or 1 to 28 days. Accordingly, the method may include administering an induction dose and determining the concentration of the antibody or antigen-binding fragment thereof in the subject's blood, plasma, or serum (e.g., the concentration is determined every six hours, every 12 hours, once per day, once every two days, once every three days, once every four days, once every five days, once every six days or once per week).

In some embodiments, the induction dose is provided to maintain a desired minimum blood, plasma, or serum concentration of the antibody or antigen-binding fragment thereof. For example, in some embodiments, it is desirable to maintain a minimum concentration (e.g., a trough concentration) of at least about 20 μg/mL (e.g., at least 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, or 250 μg/mL, 260 μg/mL, 270 μg/mL, 280 μg/mL, 290 μg/mL, 300 ug/mL (e.g., a range from about 20 ug/mL to about 150 ug/mL; from about 150 μg/mL to about 300 μg/mL; from about 150 μg/mL to about 200 μg/mL, or from about 20 μg/mL to about 300 μg/mL) of the antibody or antigen-binding fragment thereof in the blood, plasma, or serum. Accordingly, the method may include administering an induction dose and monitoring the concentration of the antibody or antigen-binding fragment thereof in the blood, plasma, or serum. Once the concentration approaches the minimum threshold (e.g., about 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, or about 150 μg/mL (or a concentration in the range of from about 20 μg/mL to about 150 μg/mL), e.g., within about 10%, 20%, 30%, 40%, or 50% of the minimum threshold), the method may include administering a further induction dose of the antibody or antigen-binding fragment thereof of, e.g., about 1 mg/kg to about 20 mg/kg (e.g., about 5 mg/kg or about 10 mg/kg), e.g., prior to establishment of maintenance dosing.

The method further includes, for example, treating the subject with one or more subsequent maintenance doses (e.g., after the induction dose(s)) of the antibody or antigen-binding fragment thereof of about 1 mg/kg to about 20 mg/kg (e.g., a dose of about 5 mg/kg or about 10 mg/kg) that is administered once every week, once every two weeks, once every three weeks, or once a month (e.g., once every two or three weeks). The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject).

In some embodiments, the maintenance dose is provided to maintain a desired minimum blood, plasma, or serum concentration of the antibody or antigen-binding fragment thereof. For example, in some embodiments, it is desirable to maintain a minimum concentration (e.g., trough concentration) of at least 150 μg/mL (e.g., at least 160 μg/mL, 170 μg/mL, 180 μg/mL, 190 μg/mL, or 200 μg/mL, e.g., from about 150 μg/mL to about 200 μg/mL) of the antibody or antigen-binding fragment thereof in the blood, plasma, or serum. Accordingly, the method may include administering an induction dose and monitoring the concentration of the antibody or antigen-binding fragment thereof in the blood, plasma, or serum. The method may include administering a maintenance dose and monitoring the concentration of the antibody or antigen-binding fragment thereof in the blood, plasma, or serum. Once the concentration approaches the minimum threshold (e.g., about 150 μg/mL or, optionally, 20 μg/mL), e.g., within 10%, 20%, 30%, 40%, or 50% of the minimum threshold), the method may include administering a maintenance dose (e.g., at a dosage of about 1 mg/kg to about 20 mg/kg, e.g., about 5 mg/kg or about 10 mg/kg) of the antibody or antigen-binding fragment thereof. The maintenance dose can be repeatedly administered following the transplant, e.g., once every week, once every two weeks, once every three weeks, or once a month (e.g., once every two or three weeks). The maintenance dose can be administered to the subject for a treatment period of at least one month (e.g., for a treatment period of at least two months, three months, four months, five months, six months, seven months, eight months, nine months, ten months, eleven months, one year, two years, three years, four years, five years, six years, seven years, eight years, nine years, ten years, or for the life of the subject). The timing of the maintenance dose and/or time between maintenance doses may be selected based on the PK profile of the anti-CD40 antibody or antigen-binding fragment thereof in transplant patients, or the PK profile in a particular subject being treated. Furthermore, the dose amount and/or timing can be titrated as needed or desired. One of skill in the art would understand that in some embodiments, the minimum concentration (e.g., trough concentration) of may fluctuate and/or temporarily dip below a desired threshold concentration, e.g., of at least 10 μg/mL (e.g., from about 10 μg/mL to about 200 μg/mL, e.g., from about 20 μg/mL, to about 150 μg/mL, e.g., at least 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, or more), of the anti-CD40 antibody or antigen-binding fragment thereof in the blood, plasma, or serum.

A blood, plasma, or serum concentration of the humanized anti-CD40 antibody or antigen-binding fragment thereof may be used to guide maintenance dosing. For example, if a serum concentration drops below a desired or predetermined threshold or range (e.g., from about 20 μg/mL to about 300 μg/mL, e.g., from about 50 μg/mL to about 200 μg/mL, from about 50 μg/mL to about 150 μg/mL, or from about 80 μg/mL to about 150 μg/mL), the subject can be administered a maintenance dose. If the serum concentration drops, for example, more than 10%, e.g., more than 20%, 30%, 40%, or more) below a predetermined threshold or range, the subject can be administered one or more “reloading dose(s)” to increase the serum concentration to a desired therapeutic level (e.g., to a concentration of from about 20 μg/mL to about 300 μg/mL, e.g., from about 50 μg/mL to about 200 μg/mL, from about 50 μg/mL to about 150 μg/mL, or from about 80 μg/mL to about 150 μg/mL). In some embodiments, the maintenance dose may be administered at a greater dosage than a prior maintenance dose (e.g., a dose of greater than 10 mg/kg or 20 mg/kg) if it is administered following a drop below a desired minimum blood, plasma, or serum concentration (e.g., below about 150 μg/mL or below about 20 μg/mL). In some embodiments, one or more maintenance dose(s) may be administered at a shorter dosing interval than the prior maintenance doses if it is administered following a drop below a desired minimum blood, plasma, or serum concentration (e.g., below about 150 μg/mL or below about 20 μg/mL).

In certain embodiments, if during induction dosing or maintenance dosing the subject has signs or symptoms of transplant rejection (e.g., elevated troponin levels or abnormal echocardiogram), the subject can be administered (i) one or more rescue dose(s) of an anti-CD40 antibody or antigen-binding fragment thereof; (ii) one or more increased dose(s) of the anti-CD40 antibody or antigen-binding fragment thereof relative to the dose level administered to the subject prior to the signs or symptoms of transplant rejection; (iii) the anti-CD40 antibody or antigen-binding fragment thereof at an increased dose frequency relative to the dose frequency administered to the subject prior to relative to the dose level administered to the subject prior to the signs or symptoms of transplant rejection, or combinations of (i)-(iii).

The blood, plasma, or serum concentration of the antibody or antigen-binding fragment thereof may be measured following administration of the antibody using an immunoassay. The immunoassay may employ plates coated with an anti-ID antibody as the capture antigen. Calibration standards and quality control (QC) samples may be prepared by diluting stock solutions of the antibody or antigen-binding fragment thereof into undiluted human serum, followed by subsequent serial dilution with undiluted human serum to the desired antibody concentration. Prepared calibrators, quality control samples and study human serum samples may be diluted to a minimum required dilution (MRD) of 1:200 using Assay Diluent prior to loading onto the pre-coated plates. The plates were incubated to allow the antibody present in the samples to bind to the target and subsequently washed to remove unbound material. Mouse anti-Hu IgG4 pFc HRP may then added to the plate to bind the antibody. The plates may be further incubated, and then may be washed to remove any unbound materials. TMB substrate may then be added to the plates and then the reaction can be stopped by adding 1N H₂SO₄. The plate may be read immediately, e.g., using a SPECTRAMAX® Plus 384 (450 nm for detection and 650 nm for background) microplate reader. The resulting OD values (detection OD at 450 nm minus background OD at 650 nm) obtained from the calibration standards may be fitted using a 4-PL logistic equation with 1/y2 to calculate the antibody concentrations in the quality control and study samples.

The calibration curve range of this method may be, for example, 8000 ng/mL to 80.0 ng/mL in 100% human serum. Calibrators outside the evaluated range of the assay at 10000 ng/mL and 40.0 ng/mL in 100% human serum may be included as anchor points to facilitate curve-fitting.

The methods described herein may include adjusting a dosage of the anti-CD40 antibody or antigen-binding fragment thereof in response to loss following administration. For example, during a transplant procedure, or another procedure following transplant, such as during the post-operative care of the subject, the subject may experience significant blood loss (e.g., as a result of the transplant surgery or another surgical procedure) that could require redosing to mitigate a reduced concentration of the anti-CD40 antibody or antigen-binding fragment thereof in the subject. Furthermore, the subject may be treated with an additional therapeutic intervention(s), such as dialysis, extracorporeal membrane oxygenation (ECMO), continuous veno-venous hemofiltration (CVVHW), mechanical ventilation, and the like, or infusion of liquids, e.g., albumin or crystalloid, that may dilute the blood, plasma, or serum concentration of the anti-CD40 antibody or antigen-binding fragment thereof. In some instances, the subject may experience a pleural effusion and, for example, may undergo thoracentesis to remove the fluid from the lungs. The anti-CD40 antibody or antigen-binding fragment thereof may be present in the pleural effusion. In other instances, the subject may require plasmapheresis, which removes plasma (and antibodies contained therein) that is separated from the blood of the subject. These therapeutic interventions, and/or the use of external instrumentation, may (directly or indirectly) reduce the blood, plasma, or serum concentration of the anti-CD40 antibody or antigen-binding fragment thereof in the subject, which may require redosing or adjustment of the dose level or dosing frequency of the anti-CD40 antibody or antigen-binding fragment thereof, or of other therapeutic agents. Accordingly, it is possible that the serum levels of the anti-CD40 antibody or antigen-binding fragment thereof are drastically reduced during or as a result of one or more of these procedures. Accordingly, the blood, plasma, or serum concentration of the anti-CD40 antibody or antigen-binding fragment thereof may be monitored prior to, during, or following one or more of these procedures. The anti-CD40 antibody or antigen-binding fragment thereof may be re-administered to the subject, or the dosage and/or dosing frequency of the anti-CD40 antibody or antigen-binding fragment thereof may be increased, as needed, to restore or increase the blood, plasma, or serum concentration of the anti-CD40 antibody or antigen-binding fragment thereof, to account for the loss of blood or other fluids, blood dilution or loss due to one or more of these procedures and/or the usage of the external instrumentation. The level of other therapeutic agents may also be monitored for loss and re-administered, as needed.

In certain methods as described herein, the subject may be treated perioperatively with a standard of care as described, e.g., in Yin et al. Chin. Med. J. 124:1928-1932, 2011, or Amer. J. Transplantation, 9: Suppl 3, 2009, S1-155, each of which are hereby incorporated by reference in their entirety.

### Transplantation

The methods described herein include performing a transplant (e.g., a cell, organ, or tissue transplant) in a subject. Methods of transplantation are well known and can be performed by a physician skilled in the art. The transplant may be an allogeneic transplant. In other embodiments, the transplant may be a xenograft transplant, e.g., a cell, organ, or tissue from a different species. The transplant may include a heart, kidney, lung, liver, pancreas, intestine, thymus, skin, eye, uterus, stem cell, bone, tendon, cornea, heart valve, nerve, vein, or a portion thereof. The xenograft transplant may a cell, organ, or tissue that has been genetically engineered, e.g., to add, modify, or remove one or more xenoreactive antigens. In some embodiments, the transplant is a xenograft transplant that includes a cell, organ (or portion thereof), or tissue from a pig, cow, horse, dog, cat, sheep, goat, non-human primate (e.g., a macaque (e.g., a rhesus macaque or a cynomolgus macaque), a baboon, a marmoset, a monkey, and a chimpanzee), or gorilla. The xenograft transplant may be a porcine heart. The xenograft transplant may be a porcine kidney. In some embodiments, the xenograft transplant includes a cell, organ or portion thereof, or tissue from a host animal that has been genetically engineered, e.g., to add, modify, or remove one or more xenoreactive antigens. Such genetically engineered animals are described, for example, in Lu, et al. Front. Immunol. 10, 3060, 2020, which is herein incorporated by reference in its entirety (see also Table 2). See also, for example, U.S. Pat. Nos. 10,912,863 and 11,179,496 and US 2018/0249688, each of which is incorporated herein by reference in their entirety.

A cell, tissue, or organ transplant can be prepared according to methods known in the art. For example, in some embodiments the organ transplant is a xenotransplant, e.g., an organ (e.g., heart) from a pig, and the host animal has been genetically engineered. The host may have been engineered to add, remove, or modify one or more xenoreactive antigens, e.g., to prevent transplant rejection. Suitable systems and methods are known in the art, e.g., as described in U.S. Pat. Nos. 11,179,496 and 10,912,863 and US Pub. No. 2018/0249688, the disclosures of which are hereby incorporated by reference in their entirety.

When a subject undergoes a transplant, significant trauma and/or bleeding to the subject's tissues or organs may occur. Accordingly, in some embodiments, certain pharmaceutical compositions (e.g., the anti-CD40 antibody or antigen-binding fragment thereof or any other therapeutic agent described herein, e.g., an anti-CD20 antibody, such as rituximab, and/or anti-thymocyte globulin and/or anti-IL-2Rα receptor antibody) may be administered, e.g., on the same day or following the transplant once the subject achieves hemostasis. A surgeon may finish the surgery once they see no obvious bleeding and then they close the skin incision when no obvious blood loss is detected. The subject may still be on pressors to support blood pressure for other hemodynamic reasons. Accordingly, hemostasis may include a visual or qualitative assessment by the surgeon. Following hemostasis, the subject may be able to leave the operating room and go to recovery.

The methods described herein can be used to treat or reduce the risk of transplant rejection. In general, transplant rejection can be characterized as hyperacute rejection, acute rejection, or chronic rejection. Hyperacute rejection occurs immediately after the transplant to about 48 hours after the transplant (e.g., when the transplant contains antigens that are mismatched relative to the recipient). Acute rejection occurs from about 2 days to about 3 months after the transplant. Chronic rejection occurs when the immune system continually attacks the transplant over time, ultimately reducing or permanently damaging the functionality of the transplant. The methods described herein can be used treat or reduce the risk of hyperacute transplant rejection. The methods described herein can also be used to treat or reduce the risk of acute transplant rejection and/or chronic transplant rejection.

Transplant rejection can be measured using methods known in the art, such as by echocardiography and/or by measuring or detecting troponin levels. Troponin is a biomarker for transplant rejection and is released by the heart's myocytes in response to injury. Thus, troponin levels can be used as a marker of cardiac function. Elevated serum levels of troponin indicate increased risk for cardiac transplant rejection.

If the subject is displaying signs or symptoms of transplant rejection, a change in the scheduled or anticipated administration of an anti-CD40 antibody or antigen-binding fragment thereof may be necessary. For example, it may be beneficial to re-load the drug and/or increase the induction or maintenance dose or increasing the frequency of dosing to reach a higher plasma concentration thereby increasing the amount of drug present in the transplanted tissue. For example, in certain embodiments, if during induction dosing or maintenance dosing the troponin levels increase, the subject can be administered (i) one or more rescue induction dose or maintenance dose, respectively, of the antibody or antigen-binding fragment in addition to the scheduled or anticipated doses; (ii) one or more increased dose(s) of the antibody or antigen-binding fragment relative to the dose level administered to the subject prior to the increase in troponin levels; (iii) the antibody or antigen-binding fragment at an increased dose frequency relative to the dose frequency administered to the subject prior to the increase in troponin levels. Similarly, in other embodiments, if during induction dosing or maintenance dosing, the subject has signs or symptoms of transplant rejection, the subject can be administered (i) a rescue induction dose or maintenance dose, respectively, of the antibody or antigen-binding fragment in addition to the scheduled or anticipated doses; (ii) one or more increased dose(s) of the antibody or antigen-binding fragment relative to the dose level administered to the subject prior to the signs or symptoms of transplant rejection; (iii) the antibody or antigen-binding fragment at an increased dose frequency relative to the dose frequency administered to the subject prior to relative to the dose level administered to the subject prior to the signs or symptoms of transplant rejection, or combinations of (i)-(iii). Other biomarkers evidencing rejection of the transplanted tissue (e.g., echocardiogram) are well known in the art.

### B Cell Reduction

The methods described herein may further include administering a therapeutic agent that depletes or reduces B cells in the subject. The therapeutic agent may be, for example, rituximab. The therapeutic agent may be administered prior to the transplant (e.g., from about 1 hour to about 24, e.g., about 3 hours to about 24 hours, such as at about 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant). The therapeutic agent may be administered after the transplant (e.g., one day, two days, three days, four days, five days, six days, one week, two weeks, three weeks, and/or four weeks after the transplant). In some embodiments, the therapeutic agent is administered for a treatment period of one day, two days, three days, four days, five days, six days, one week, two weeks, three weeks, or four weeks. In some embodiments, the therapeutic agent is administered daily, weekly, or once every other week for a treatment period of one week, two weeks, three weeks, or four weeks. In some embodiments, the therapeutic agent (e.g., rituximab) is administered at a dosage of about 1 mg/kg to about 40 mg/kg (e.g., about 10 mg/kg). In some embodiments, the therapeutic agent is administered intravenously. In some embodiments, the rituximab is administered at a dosage of about 250 mg/m² to about 500 mg/m², e.g., a dosage of about 375 mg/m².

### T Cell Reduction

The methods described herein may further include administering a therapeutic agent that depletes or reduces T cells in the subject. The therapeutic agent may be, for example, anti-thymocyte globulin, e.g., THYMOGLOBULIN® or ATGAM®. The therapeutic agent may be administered prior to the transplant (e.g., from about 1 hour to about 24 hours prior to the transplant, e.g., about 3 hours to about 24 hours, such as about 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours prior to the transplant). In some embodiments, the therapeutic agent is administered on the same day as the transplant. The therapeutic agent may be administered after the transplant (e.g., one day, two days, three days, four days, five days, six days, one week, two weeks, three weeks, and/or four weeks after the transplant). In some embodiments, the therapeutic agent is administered for a treatment period of one day, two days, three days, four days, five days, six days, one week, two weeks, three weeks, or four weeks. In some embodiments, the therapeutic agent is administered daily, weekly, or once every other week for a treatment period of one week, two weeks, three weeks, or four weeks. In some embodiments, the therapeutic agent is administered within about 1 hour to about 24 hours following the transplant and once the subject achieves hemostasis.

In some embodiments, the therapeutic agent (e.g., anti-thymocyte globulin, e.g., THYMOGLOBULIN® or ATGAM®, or anti-IL-2Rα receptor antibody) is administered at a dosage of about 1 mg/kg to about 10 mg/kg, e.g., about 2 mg/kg or about 5 mg/kg. In some embodiments, the therapeutic agent is THYMOGLOBULIN® and is administered at a dosage of about 2 mg/kg. In some embodiments, the therapeutic agent is ATGAM® and is administered at a dosage of about 5 mg/kg. The therapeutic agent may be administered intravenously.

Additional Therapeutic Agents

The methods described herein may further include administration of an additional therapeutic agent, e.g., prior to, on the same day as, or following the transplant. The method may include administration of any drug required to maintain suppression of the immune system or promote recovery following surgery. The method may further include administering one or more of a steroid (e.g., methylprednisolone), an antihistamine (e.g., diphenhydramine), an H2 receptor blocker (e.g., famotidine), an antiviral agent (e.g., ganciclovir), a complement inhibitor (e.g., a C1 esterase inhibitor), an immunosuppressive agent (e.g., an anti-IL-6R antibody, such as tocilizumab, and/or mycophenolate mofetil), an anti-inflammatory agent (e.g., TNFα protein, such as etanercept, or a non-steroidal anti-inflammatory agent (NSAID, such as aspirin and naproxen), an anticoagulant (e.g., heparin and aspirin or other known agent), and an antibiotic (e.g., ceftriaxone or other known agent). For example, the method may include administering one or more of methylprednisolone, diphenhydramine, famotidine, ganciclovir, a C1 esterase inhibitor, tocilizumab, etanercept, mycophenolate mofetil, heparin, aspirin, and ceftriaxone.

The methods described herein may further include administering a steroid, such as methylprednisolone, or other steroid known in the art. In some embodiments, the methylprednisolone is administered once per day or twice per day. The methylprednisolone may be administered at a dosage of about 2 mg/kg. The methylprednisolone may be administered prior to the transplant. The methylprednisolone may be administered one day prior to the transplant. The methylprednisolone may be administered on the same day as the transplant. The methylprednisolone may be administered after the transplant. The methylprednisolone may be administered one day, two days, three days, four days, five days, six days, one week, two weeks, three weeks, four weeks, two months, three months, four months, five months, and/or six months after the transplant. The methylprednisolone may be administered for a treatment period of 2 months, seven weeks, six weeks, five weeks, four weeks, three weeks, two weeks, one week, six days, five days, four days, three days, two days, or one day.

The methods described herein may further include administering an antihistamine, such as diphenhydramine, or other antihistamine known in the art. The diphenhydramine may be administered at a dosage of about 50 mg. The diphenhydramine may be administered intravenously. The diphenhydramine may be administered prior to the transplant. The diphenhydramine may be administered one day prior to the transplant. The diphenhydramine may be administered on the same day as the transplant. The diphenhydramine may be administered after the transplant. The diphenhydramine may be administered one day, two days, three days, four days, five days, six days, one week, two weeks, three weeks, and/or four weeks after the transplant. The diphenhydramine may be administered for a treatment period of 2 months, seven weeks, six weeks, five weeks, four weeks, three weeks, two weeks, one week, six days, five days, four days, three days, two days, or one day.

The methods described herein may further include administering an H2 receptor blocker, such as famotidine, or other H2 receptor blocker known in the art. The famotidine may be administered at a dosage of about 20 mg. The famotidine may be administered once per day or twice per day. The famotidine may be administered prior to the transplant. The famotidine may be administered one day prior to the transplant. The famotidine may be administered on the same day as the transplant. The famotidine may be administered after the transplant. The famotidine may be administered one day, two days, three days, four days, five days, six days, one week, two weeks, three weeks, four weeks, five weeks, six weeks, seven weeks, and/or eight weeks after the transplant. The famotidine may be administered for a treatment period of 2 months, seven weeks, six weeks, five weeks, four weeks, three weeks, two weeks, one week, six days, five days, four days, three days, two days, or one day.

The methods described herein may further include administering an antiviral agent, such as ganciclovir, or another antiviral agent known in the art. The ganciclovir may be administered at a dosage of about 5 mg/kg. The ganciclovir may be administered prior to the transplant. The ganciclovir may be administered one day prior to the transplant. The ganciclovir may be administered on the same day as the transplant. The ganciclovir may be administered after the transplant. The ganciclovir may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The ganciclovir may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The ganciclovir may be administered daily for a treatment period of at least one year, two years, three years, four years, five years, six years, seven years, eight years, nine years, ten years, or for the life of the subject.

The methods described herein may further include administering a complement inhibitor, such as C1 esterase inhibitor, or other complement inhibitor known in the art. The C1 esterase inhibitor may be administered at a dosage of about 20 U/kg. The C1 esterase inhibitor may be administered prior to the transplant. The C1 esterase inhibitor may be administered one day prior to the transplant. The C1 esterase inhibitor may be administered on the same day as the transplant. The C1 esterase inhibitor may be administered after the transplant. The C1 esterase inhibitor may be administered one day, two days, three days, four days, five days, six days, and/or one week after the transplant.

The methods described herein may further include administering an immunosuppressive agent, such as tocilizumab, or another immunosuppressive agent known in the art. The tocilizumab may be administered at a dosage of about 8 mg/kg. The tocilizumab may be administered intravenously. The tocilizumab may be administered on the same day as the transplant. The tocilizumab may be administered after the transplant. The tocilizumab may be administered one month, two months, three months, four months, five months, six months, seven months, eight months, nine months, ten months, eleven months, and/or twelve months after the transplant. The tocilizumab may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The tocilizumab may be administered monthly for a treatment period of at least one year, two years, three years, four years, five years, six years, seven years, eight years, nine years, ten years, or for the life of the subject.

The methods described herein may further include administering an anti-inflammatory agent, such as etanercept or another anti-inflammatory agent known in the art. The etanercept may be administered at a dosage of about 0.7 IU/kg. The etanercept may be administered subcutaneously. The etanercept may be administered on the same day as the transplant. The etanercept may be administered after the transplant. The etanercept may be administered one week, two weeks, three weeks, one month, two months, three months, four months, five months, six months, seven months, eight months, nine months, ten months, eleven months, and/or twelve months after the transplant. The etanercept may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The etanercept may be administered weekly for a treatment period of at least one year, two years, three years, four years, five years, six years, seven years, eight years, nine years, ten years, or for the life of the subject.

The methods described herein may further include administering an immunosuppressive agent, such as mycophenolate mofetil, or another immunosuppressive agent known in the art. The mycophenolate mofetil may be administered at a dosage of about 20 mg/kg. The mycophenolate mofetil may be administered intravenously. The mycophenolate mofetil may be administered once per day or twice per day. The mycophenolate mofetil may be administered on the same day as the transplant. The mycophenolate mofetil may be administered after the transplant. The mycophenolate mofetil may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The mycophenolate mofetil may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The mycophenolate mofetil may be administered daily for a treatment period of at least one year, two years, three years, four years, five years, six years, seven years, eight years, nine years, ten years, or for the life of the subject.

The methods described herein may further include administering an anticoagulant, such as heparin, or another anticoagulant known in the art. The heparin may be administered on the same day as the transplant. The heparin may be administered after the transplant. The heparin may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The heparin may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The heparin may be administered daily for a treatment period of at least one year, two years, three years, four years, five years, six years, seven years, eight years, nine years, ten years, or for the life of the subject.

The methods described herein may further include administering a non-steroidal anti-inflammatory agent (NSAID), such as aspirin. The aspirin may be administered at a dosage of about 81 mg. The aspirin may be administered after the transplant. The aspirin may be administered one day, two days, three days, four days, five days, six days, one week, two weeks, three weeks, four weeks, five weeks, six weeks, seven weeks, eight weeks, three months, four months, five months, six months, seven months, eight months, nine months, ten months, eleven months, one year, two years, three years, four years, five years, six years, seven years, eight years, nine years, and/or ten years after the transplant. The aspirin may be administered for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject. The aspirin may be administered daily for a treatment period of at least one year, two years, three years, four years, five years, six years, seven years, eight years, nine years, ten years, or for the life of the subject.

The methods described herein may further include administering an antibiotic, such as ceftriaxone, or another antibiotic known in the art. The ceftriaxone may be administered at a dosage of about 50 mg/kg. The ceftriaxone may be administered after the transplant. The ceftriaxone may be administered one day, two days, three days, four days, five days, six days, one week, and/or two weeks after the transplant. The ceftriaxone may be administered for a treatment period of one day, two days, three days, four days, five days, six days, one week, or two weeks. The ceftriaxone may be administered daily for a treatment period of one week.

### Pharmaceutical Compositions

Featured are methods of treating or reducing the risk of transplant rejection or increasing a duration of time before transplant rejection occurs by administering a pharmaceutical composition containing an anti-CD40 antibody or antigen-binding fragment thereof, such as those described herein (e.g., an antibody or antigen-binding fragment thereof that includes a heavy chain variable region including a CDRH1, CDRH2, and CDRH3 as set forth SEQ ID Nos: 1-3, respectively, and a light chain variable region including a CDRL1, CDRL2, and CDRL3 as set forth in SEQ ID Nos: 4-6, respectively, such as an antibody or antigen-binding fragment thereof with a heavy chain that includes an amino acid sequence having at least 85% (e.g., at least 90%, 95%, 97%, 99%, or 100%) sequence identity to SEQ ID NO: 7 or 9, and a light chain that includes an amino acid sequence having at least 85% (e.g., at least 90%, 95%, 97%, 99%, or 100%) sequence identity to SEQ ID NO: 8 or 10). The antibody or antigen-binding fragment thereof can be formulated in a pharmaceutical composition in a concentration of about 50 to about 300 mg/mL, and optionally, in a volume of about 0.5 mL to about 2.0 mL (e.g., about 1.0 mL).

The antibody or antigen-binding fragment thereof (e.g., KPL-404) may be incorporated into a pharmaceutical composition (e.g., in a concentration of about 50 mg/mL to about 300 mg/mL, such as about 100 mg/mL or about 200 mg/mL). The composition may include a polar excipient, for example, a sugar, a polyol, or an amino acid. In some embodiments, the sugar is, for example, sucrose, trehalose, fructose, lactose, dextrose, or mannitol. In some embodiments, the polyol is, for example, polyethylene glycol or sorbitol. In some embodiments, the amino acid is one or more of alanine, arginine, aspartic acid, asparagine, carnitine, citrulline, ornithine, glycine, glutamic acid, glutamine, glycine, isoleucine, leucine, lysine, methionine, phenylalanine, proline, serine, threonine, tyrosine, and valine. The composition may also include acetate may include, for example, sodium acetate (e.g., in salt form) or acetate in its ionic form. In some embodiments, the composition includes, for example, sucrose, arginine, glutamate, or sorbitol, or a combination thereof. In some embodiments, the excipient is sucrose. In some embodiments the polar excipient is arginine. In some embodiments the excipient is glutamate. In some embodiments, the excipient is a mixture of arginine and glutamate. In some embodiments, the excipient is sorbitol.

The pharmaceutical composition can include, e.g., an amount of an anti-CD40 antibody or antigen-binding fragment thereof described herein (e.g., KPL-404), such as, e.g., about 50 mg/mL to about 300 mg/mL).

The antibody or antigen-binding fragment thereof may be provided in a container including a pharmaceutical composition as described herein. Suitable containers include, for example, bottles, vials, syringes, and test tubes. The containers may be formed from a variety of materials such as glass or plastic. The container holds a pharmaceutical composition as described herein, e.g., that is effective for treating the condition, and may have a sterile access port. For example, the container may be an intravenous solution bag or a vial having a stopper pierceable by a hypodermic injection needle. The label on or associated with the container indicates that the composition is used for treating the condition of choice. The kit may further include a second container with a pharmaceutically acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The antibody or antigen-binding fragment thereof may be present in the container at a concentration of from about 50 mg/mL to about 300 mg/mL (e.g., 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, or 300 mg/mL), such as at a concentration of about 100 mg/mL or about 200 mg/mL.

The composition may be formulated in a single use vial with about 1.0 mL or 2.0 mL extractable volume. The composition can be formulated in a volume of about 0.1 mL to about 2.0 mL (e.g., 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, or 2.0 mL), such as a volume of about 1.0 mL.

### EXAMPLES

The following examples of specific aspects for carrying out the present disclosure are offered for illustrative purposes only and are not intended to limit the scope of the present disclosure in any way.

### Example 1

An anti-CD40 antibody, KPL-404, can be used to treat or reduce the risk of (e.g., inhibit or prevent) xenotransplantation rejection, and the treatment may be performed in conjunction with additional immunosuppressive treatments. The method was performed in a pig-to-baboon orthotopic cardiac xenotransplantation model using a baboon that received transplantation of a heart from a pig that was genetically modified to include 10 gene modifications that eliminated expression of antigens that are known to provoke humoral and cellular immune responses.

### Animal Model

A conventional Papio anubis baboon weighing 15-30 kg was screened to eliminate specific pathogens of interest and used as a recipient. A weight-matched German Landrace pig, with the genetics described in Table 2, was used as a cardiac donor (supplied by Revivicor Inc. Blacksburg, Va.). An orthotopic porcine heart transplant was performed in the baboon.

TABLE 2

| Gene | Description |
|---|---|
| Gal KO | Inactivation or knock out (KO) of the GGTA1 gene that is responsible for the |
| | expression of the antigen α 1,3-galactose (α-gal). |
| β4GalNT2 | Inactivation or KO of the β1,4 N-acetylgalactosaminyltransferease-2 that is |
| KO | responsible for the expression of carbohydrate antigen SDa |
| CMAH-KO | Inactivation or KO of cytidine monophosphate-N-acetyl neuraminic acid hydroxylase |
| | gene that is responsible for the expression of the antigen N-glycolylneuraminic acid |
| | (Neu5Gc). |
| GHr KO | Inactivation or KO of the porcine growth hormone receptor gene that is responsible |
| | for regulating growth of the animal. |
| CD46-DAF | Human complement pathway inhibitor gene, CD46. Overexpression of the human |
| | gene in the pig will inhibit complement mediated cell lysis. |
| | Human decay accelerating factor (DAF) gene is a complement pathway inhibitor |
| | gene. Overexpression of this gene in the pig will inhibit complement mediated cell |
| | lysis. |
| | Bi-cistronic transgene vector in which CD46 and DAF are linked by a 2A sequence to |
| | permit expression by a single Cytomegalovirus enhancer, chicken beta-Actin and the |
| | rabbit beta-Globin (CAG) promoter. This vector is flanked with homology arms to |
| | facilitate targeted, mono- allelic (CD46-DAFM) or bi-allelic (CD46-DAF_{B}) insertion |
| | by homology directed repair (HDR) into a landing pad directed to pPL-657. |
| TM-EPCR- | The human THBD gene expresses the protein thrombomodulin, TM, that inhibits |
| CD47-H01 | coagulation, thrombosis and platelet aggregation in vascular and microvascular |
| | endothelium of transplanted organs. |
| | The endothelial cell protein C receptor (EPCR) protein expressed by the human |
| | PROCR gene. EPCR has anticoagulant and anti-inflammatory activity when |
| | expressed in transplanted organs. |
| | The human CD47 gene expresses the CD47 protein that inhibits macrophage |
| | responses in transplanted organs. |
| | The human hemoxygenase-1 (HO1) gene expresses heme oxygenase 1 that |
| | provides anti-inflammatory characteristics to transplanted organs. |
| | Tetra-cistronic transgene vector composed of two bi-cistrons. In the first, human TM |
| | and EPCR are linked by a 2A sequence and expressed by a single porcine TM |
| | promoter. Linked downstream to this is a second bi-cistron containing CD47 and |
| | HO1, linked by a 2A sequence and driven by a single CAG promoter. This vector is |
| | flanked with homology arms to facilitate targeted, mono- allelic (TM-EPCR-CD47- |
| | HO1M) or bi-allelic (TM-EPCR-CD47-HO1B) insertion into landing pads on the |
| | CMAH locus by HDR. |

### Immunosuppressive Regimen

The immunosuppressive regimen for the recipient baboon included induction therapy and maintenance therapy, as shown in Table 3. Induction immunotherapy includes anti-thymocyte globulin (THYMOGLOBULIN®), anti-CD20 antibody (RITUXAN®) for T and B cell suppression and humanized anti-CD40 antibody (KPL-404); for blocking the CD40/CD154 co-stimulation pathway, and anti-inflammatory drugs, Tociluzimab and Etanercept. C1 Esterase Inhibitor (Berinert) was used to inhibit complement activation. Maintenance immunotherapy included administration of Mycophenolate Mofetil (MMF) and the anti-CD40 antibody (KPL-404) and the anti-inflammatory drugs, Tociluzimab and Etanercept. The baboon received continuous heparin infusion to keep the activated clotting time (ACT) level twice the baseline. Ganciclovir was administered daily to prevent Cytomegalovirus (CMV) infection. Other medications include Epogen and Cephazolin.

TABLE 3
Immunosuppression Regimen for cardiac Xenotransplantation

| | Drugs | Dose | | Timing |
|---|---|---|---|---|
| Induction: | Rituxan (Anti CD20) | 19 | mg/Kg | Pre op days −7, 0 |
| | ATGAM | 5 | mg/Kg | Pre op days −2 & −1 |
| | KPL-404 (Anti CD40 | 25-50 | mg/Kg | Pre op days −1 & 0 |
| | antibody) | | | |
| | CVF or BERINERT | 50-100 | U/Kg or | Pre op days −1, 0 |
| | | 17.5 | mg/kg | |
| | Tocilizumab | 8 | mg/kg | Day 0 |
| | Etanercept | 0.7 | mg/kg | Day 0 |
| | Steroids | 25 | mg/kg | Days −7, −2, −1; |
| | | | | 20 mg/kg day 0 |
| Maintenance: | Rituxan (Anti CD20) | 19 | mg/Kg | Days 7 and 14 |
| | CVF or BERINERT | 50-100 | U/Kg or | Day 1 |
| | | 17.5 | mg/kg | |
| | KPL-404 (Anti CD40 | 25-50 | mg/Kg | Days 4, 7, 10, 14, 19, |
| | (antibody) | | | q weekly |
| | MMF | 20 | mg/Kg/2 hr | BID daily |
| | | IV infusion | | |
| | Tocilizumab | 8 | mg/kg | Once a month after |
| | | | | Transplantation (Days |
| | | | | 30, 60, 90 and so on) |
| | Etanercept | 0.7 | mg/kg | Once Weekly after |
| | | | | Transplantation |
| | Aspirin | 40 | mg | Day 1-6, then tapered off |
| | Heparin | Maintain ACT | | Continuous infusion |
| | | 2 × baseline | | |
| | Steroids | 5 | mg/kg | Daily. Tapered off in 2 |
| | | | | months |
| Supportive: | Ganciclovir | 5 | mg/Kg/day | Daily |
| | Cefazolin | 250 | mg/Kg | BID for 7 days |
| | Epogen | 200 | U/Kg | Daily for 7 days and then |
| | | | | weekly |

### Transplant Procedure

Preoperative transthoracic echocardiograms (TTEs) were conducted to ensure adequate cardiac function free of anatomic abnormalities deemed unsatisfactory for transplantation. Donor swine heart procurement was performed using 30 cc/kg of blood cardioplegia with XVIVO© heart solution (XHS) for induction preservation (XVIVO© Perfusion, Gothenburg, Sweden). Cardiac preservation was performed using an XVIVO© Perfusion system with XHS cardioplegia at 8° C., maintaining a perfusion pressure of 20 mmHg in the aortic root and physiological pH (7.2-7.6). Life-supporting heart xenotransplantation was performed after placing the baboon recipient onto Aorto-bicaval cardiopulmonary bypass (CPB). The donor porcine xenograft was placed in the orthotopic position after native heart explantation using a biatrial anastomosis technique.

### Results

Graft function was monitored via telemetry and periodic echocardiography. Blood work was performed weekly, and Troponin, ACT, chemistry and CBC were measured. Endomyocardial biopsies were performed periodically. Rejection was determined by elevated troponin levels, drop in pressures on telemetry, and loss of graft function on echocardiography. Blood work was also performed whenever the baboon was anesthetized or for any reason. Rejection, if present, was assessed by histology and immunohistochemistry.

At each time blood is collected for the blood work noted above, separate blood samples were collected in accordance with the routine methods and stored for later evaluation using a Pharmacokinetics Assay and Anti-Drug Antibody (ADA) Assay. Blood samples were also assessed for PK and ADA analysis.

As of 33 days post-surgery, the baboon is recovering with no signs of transplant rejection, as measured by echnocardiography and Troponin levels, which is are markers of cardiac function. The echocardiogram showed normal function, and the troponin plasma levels were normal.

The treatment did not completely abolish the T-cells in the recipient baboon. ATGAM® brings levels of T-cells down to about 10% of the pre-treatment levels. Therefore, anti-CD40 treatment and mycophenolate mofetil (MMF) treatment, an antiproliferative agent, were used to attenuate T-cell function, such as T-cell cytotoxicity (e.g., via CD40 blockade on antigen presenting cells). Similarly, THYMOGLOBULIN® would be expected to reduce T cells to about 1-10% in an equivalent procedure performed in humans. Thus, the expectation is that anti-CD40 treatment in a human subject that undergoes a transplant (whether allogeneic or xenogeneic) would mitigate transplant rejection caused by any remaining B- or T-cells, given that the treatments do not completely ablate these cells in the subject. Anti-CD40 treatment would also be expected to reduce or inhibit transplant rejection as the B- and T-cells begin to regenerate after B- and T-cell reducing agent treatment is ceased or titrated downward.

Although the baboon received a high dose of the KPL-404 antibody (e.g., 25-50 mg/kg), this was done to ensure an excess of the antibody over what would likely be required to treat or reduce the risk of transplant rejection. Accordingly, it would be understood that a reduction in the risk of transplant rejection could be achieved in a subject (e.g., a human subject) that receives a dosage of KPL-404 of less than 25-50 mg/kg, such as a dosage of about 1 mg/kg to about 20 mg/kg (e.g., 5 mg/kg or 10 mg/kg).

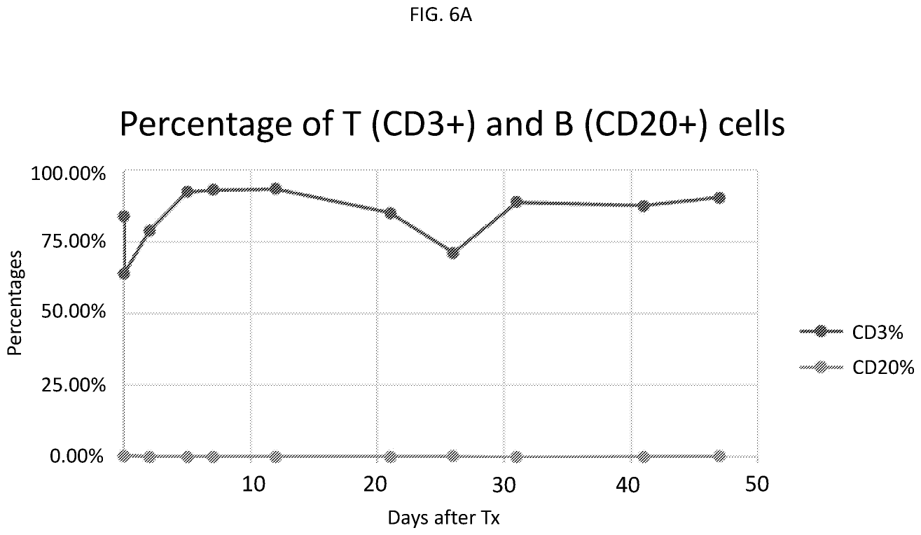

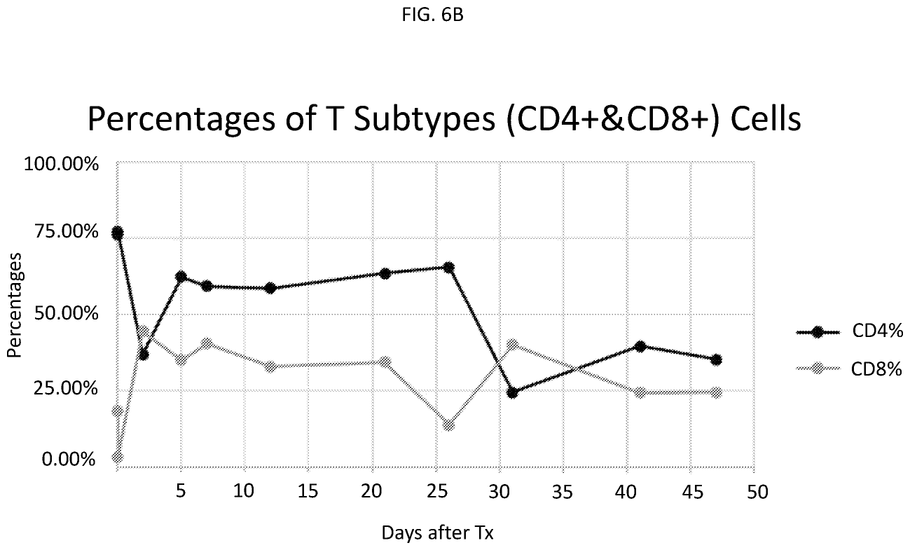

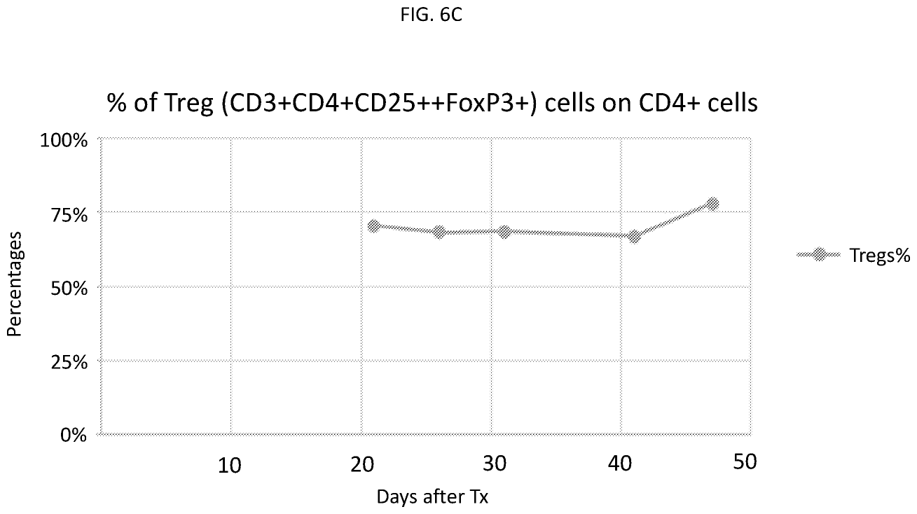

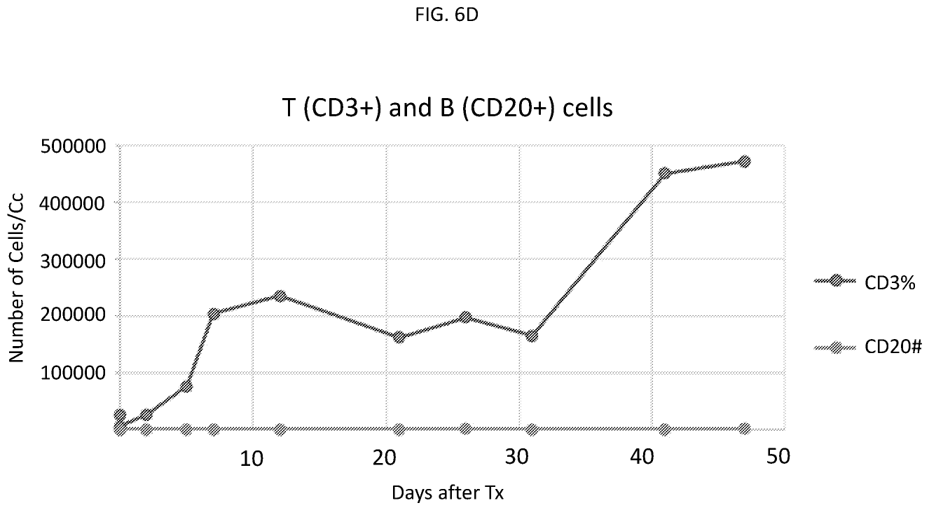

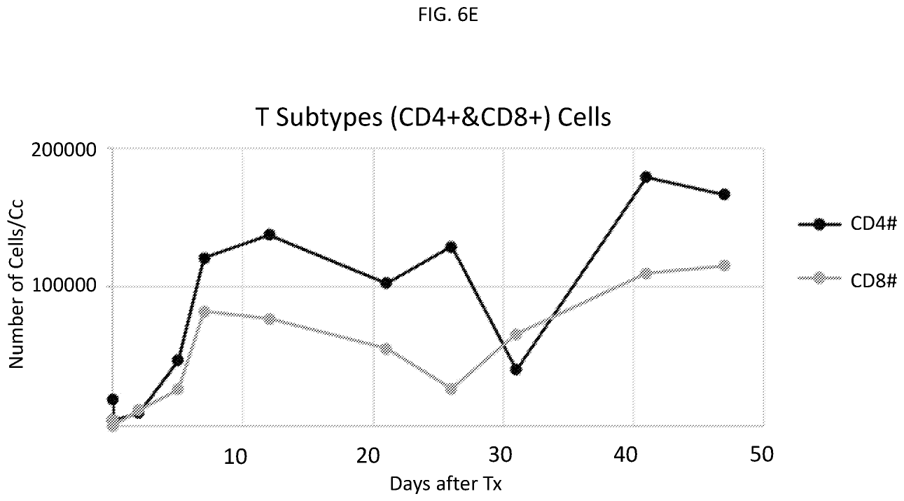

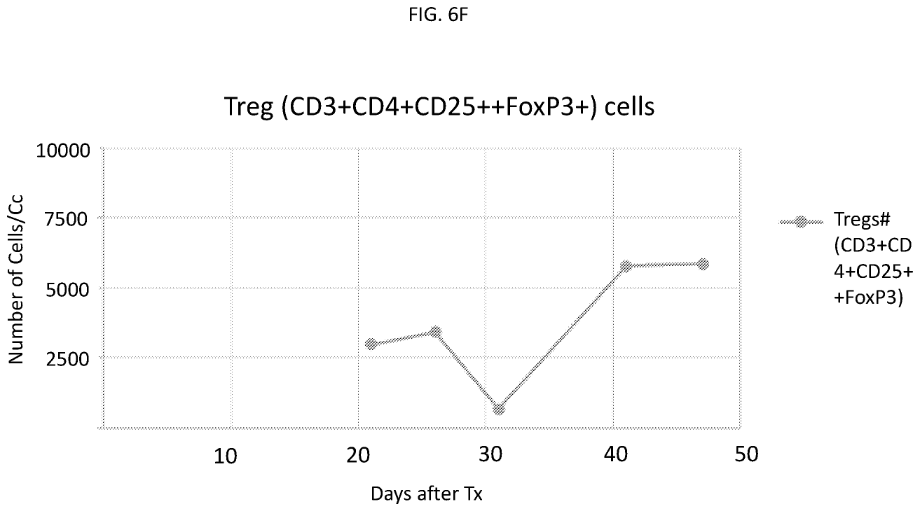

B cell and T cell counts were assessed following the transplant. FIGS. 6A-6C show percentages of B cells and T cells counts in peripheral blood relative to total blood lymphocyte count, and FIGS. 6D-6F show absolute numbers of B and T cells in peripheral blood. These graphs indicate that T cells were still present in the animal at the time of transplant and T-cell count steadily increased over time soon after transplant, while B cell counts remained low after transplant, possibly due to the action of the B-cell depleting antibody, rituximab.

### Example 2

We describe a 57-year-old male with non-ischemic cardiomyopathy dependent on veno-arterial extracorporeal membrane oxygenation without candidacy for standard therapeutics including a traditional allograft, who was transplanted with a heart from a genetically modified pig source animal with 10 individual gene edits. Immunosuppression was based on CD40 blockade. The patient was able to wean from ECMO and the xenograft functioned normally for 48 days when it developed acute biventricular thickening and diastolic failure. Support was withdrawn on post-operative day (POD) 60. At autopsy the xenograft was edematous, nearly doubling in weight. Histology revealed scattered myocyte necrosis, interstitial edema, red blood cell extravasation and prominent endothelial nuclei, without evidence of microvascular thrombosis, not consistent with typical transplant rejection.

The patient presented with chronic mild thrombocytopenia, hypertension, non-ischemic cardiomyopathy (NICM) and previous mitral valve repair. He required hospitalization for severe heart failure with a left ventricular ejection fraction (LVEF) of 10%. His care was escalated to include multiple intravenous inotropes and the placement of an intraaortic balloon pump was added on hospital day (HOD) 11. Despite this, he suffered multiple ventricular arrythmias with arrests requiring resuscitation and was emergently placed on peripheral venoarterial (VA) extracorporeal membrane oxygenation (ECMO) on HOD 23.

The patient did not meet criteria for allotransplantation or for a ventricular assist device (VAD) due to severe sarcopenia and a three-week non-ambulatory status. There was also concern about the effectiveness of a VAD due to his severe biventricular failure with ventricular arrythmias. Given single organ failure, the patient was considered for experimental xenotransplantation. Despite his biventricular failure, the patient exhibited preserved renal function and required only intermittent nasal cannula for mild hypoxia. His pre-transplant ICU course was notable for adrenal insufficiency, gastrointestinal bleeding, bacteremia, which cleared with antimicrobial therapy, and drug induced leukopenia. He underwent cardiac xenotransplantation from a genetically modified pig source animal. The human subject received a porcine heart xenograft transplant generally according to the following protocol.

TABLE 4
Immunosuppresion Regimen for cardiac Xenotransplantation: Induction

| | | | | | | Timing |
|---|---|---|---|---|---|---|
| | Day | Drugs | Dose | | Infusion Duration | Example* |
| Induction: | −1 | Methylprednisolone | 125 | mg | Administers 60 mins before rituximab | 9:00 am |
| | | | | | Administers as IV push over 3-15 minutes | |
| | | Rituximab (brand)* | 375 | mg/m* | Start infusion at a rate of 50 mg/hour, if there is | 10:00 am |
| | | NOT one biosimilar | (dose: 800 mg, rounded | | no infusion-related reaction, increase the rate by | |
| | | | to nearest 100 mg per | | 50 mg/hour increments every 30 minutes, to a | |
| | | | protocol) | | maximum rate of 400 mg/hour. | |
| | | | | | Infusion duration~3 hours. | |
| | | | | | BUD: 24 hours | |
| | | Berinert | 20 | U/kg | Administer at a rate of 4 mL/min. | 2:00 pm |
| | | | (dose: 1500 units, | | Duration of infusion: 7.5 mins | |
| | | | rounded to nearest vial | | BUD: 8 hours | |
| | | | size) | | | |
| | | KPL-404 | 10 | mg/Kg | Duration of infusion: 1 hour | 17:00 |
| | | | (dose: 850 mg) | | Needs to be administered a minimum of 3 hours | |
| | | | | | AFTER rituximab completion. | |
| | | | | | Needs to be administered a minimum of 6 hours | |
| | | | | | BEFORE OR. | |
| | | OR DATE | | | | 7:00 am |
| | 0 | Methylprednisolone | 1000 | mg | IV piggy back, to be administered in the OR | Intra-op |
| | | KPL-404 | 10 | mg/Kg | Administer over 1 hour. | 15:00 |
| | | | (dose: 850 mg) | | BUD: 4 hour | |
| | | | | | To be administered post-operatively in the CSICU. | |
| | | Berinert (C1 esterase | 20 | U/kg | Administer at a rate of 4 mL/min. | 16:00 |
| | | inhibitor) | (1500 units, rounded to | | Duration of infusion: 7.5 mins | |
| | | | nearest vial size) | | | |
| | | Methylprednisolone | 125 | mg IV | Administer as IV push over 3-15 minutes | 16:30 |
| | | | | | To be administered as pre-medication for | |
| | | | | | Thymoglobulin. | |
| | | | | | Should be administered 1 hour prior to | |
| | | | | | Thymoglobulin infusion. | |
| | | Thymoglobulin** | 2 | mg/Kg | Duration of infusion: 6 hours | 17:30 |
| | | | 2 mg/Kg | | To be administered in the CSICU post-operatively | |
| | | | (150 mg, dosed | | Dose to be adjusted | |
| | | | off IBW) | | based on WBC, platelt, CO3. | |
| | | Berinert (C1 esterase | 20 | U/Kg | Administer at a rate of 4 mL/min | 18:00 |
| | | inhibitor) | (1500 units, rounded | | Duration of infusion: 7.5 mins | |
| | | | to nearest vial size) | | | |
| | 1 | Thymoglobulin** | 2 | mg/Kg | Duration of infusion: 5 hours. | 11:00 |

| | |
|---|---|
| (150 mg, dosed | Dose to be adjusted |
| off IBW) | based on WBC, platelt, CO3) |

*Rituximab to be pre-medicated with methylprednisolone, famotidine, diphenhydramine, and acetaminophen.
**THYMOGLOBULIN ® to be pre-medicated with methylprednisolone, acetaminophen, and diphenhydramine.
±Timing: times in column are meant to be illustrative of order of administration sequence, timing will change based on operating room (OR) date/time
BUD, beyond use date; Day −1: day prior to surgery; Day 0: day of surgery; Day 1: day 1 after surgery; IBW = ideal body weight; BUD = beyond use date; CSICU = cardiac surgery ICU

TABLE 5
Immunosuppression Regimen for sardiasXenotransplantation: Maintenance

| | Drug | Dose | Date of Administration/Frequensy |
|---|---|---|---|
| Maintenance: | Rituximab (brand)* | 375 mg/rn² | Once weekly with next dose due POD7 |
| | NOT the biosimilar | (dose: 800 mg, rounded to nearest 100 | Dosing to be driven by B-cell monitoring |
| | | mg per protocol) | |
| | KPL-404 | 10 mg/kg | Once every 14 days starting POD14 |
| | | (dose: 850 mg) | |
| | Mycophenolate | 500 mg IV | Twice daily |
| | mofetil | (Dose will be up-titrated to 1000 mg BID | To be ordered post-operatively upon arrival |
| | | transition to suspension via enteral tube | to the CSICU. |
| | | when there is no concern for ileus) | |
| | | BUD: 24 hours | |
| Supportive: | Aspirin | 81 mg | Once daily |
| | | | To be started post-operative when no |
| | | | concern for bleeding |
| | Therapeutic | To be driven by the CSICU team based on | |
| | anticoagulation | clinical status | |
| | Famotidine | 20 mg twice daily | Dose to be adjusted off renal function |
| | Gancidovir | 5 mg/kg IV | Daily (dose to be adjusted off renal function) |
| | Meropenem | Dose to be adjusted off renal function per | Duration TBD based on post-op course |
| | | CSICU team | |
| | Vancomycin | Dose to be adjusted off renal function per | Duration TBD based on post-op course |
| | | CSICU team | |

*Rituximab to be pre-medicated with methylprednisolone, famotidine, diphenhydramine, and acetaminophen.

TABLE 6
Post Operative Steroid Taper

| Post-Operative Day | Methylprednisolone Dose |
|---|---|
| POD0 | Methylprednisolone 125 mg IV |
| | every 8 hours × 3 doses |
| | To be ordered and administered |
| | post-operatively upon arrival |
| | to the CSICU |
| POD1 | 80 mg IV × 1 dose |
| POD2 | 80 mg IV × 1 dose |
| POD3 | 80 mg IV × 1 dose |
| POD4 | 60 mg IV × 1 dose |
| POD5 | 60 mg IV × 1 dose |
| POD6 | 60 mg IV × 1 dose |
| POD7 | 40 mg IV × 1 dose |
| POD8-onward | 40 mg IV daily (can be converted |
| | to prednisone 40 mg PO once |
| | daily if tolerating enteral feeds) |

### Methods

Genetically Engineered Pig Source Animal

The pig was supplied by Revivicor, Inc. and was clonally derived from fibroblasts, a cell line that included 10 gene edits (see, e.g., U.S. Pat. Nos. 10,912,863 and 11,179,496 and US 2018/0249688, each of which is incorporated herein by reference in their entirety) to render the cardiac xenograft more compatible for transplantation into a human.

Preservation of the Xenograft

The pig heart was procured from the source animal. Non-ischemic perfusion of the 328 gm pig heart using the XVIVO system lasted 114 minutes. Perfusate was cooled to 8 degrees centigrade. It consisted of 4 parts Steen cardiac solution mixed with one part human blood which was type-matched to the recipient. Perfusion was fixed at 20 mmHg in the aortic root. Flow increased from 148 cc/min to 194 cc/min suggesting coronary relaxation. Total cold ischemia time was 150 minutes. The implant required 63 minutes and was interrupted three times for intermittent cardioplegia with XVIVO perfusate harvested from the circuit. An additional circulatory arrest time of 13 minutes was required after initial cardiac reanimation for repair of a Type A aortic dissection caused by the aortic cross clamp. After both the first and second circulatory arrest and rewarming, the heart began beating spontaneously with only temporary need of epicardial pacing.

Surgical Technique

The XVIVO system was transported into the hospital's operating room from the laboratory operating room. The redo sternotomy in the anesthetized patient was delayed until the donor heart was delivered. Implantation was performed using a biatrial anastomosis followed by separation from cardiopulmonary bypass following the xenotransplantation.

Immunosuppression and Monitoring

Rituximab and anti-thymocyte globulin (ATG) were used for B and T cell depletion, respectively, and complement C1 esterase inhibitor (Berinert, King of Prussia, Pa.) was used for complement inhibition. Humanized monoclonal antibody (KPL-404, Kiniksa Pharmaceuticals, Hamilton, Bermuda) was administered to block CD40 co-stimulation. A pulse-dose of methylprednisolone (1,000 mg, day of xenotransplant) was also administered. Maintenance immunosuppression included mycophenolate mofetil (MMF), KPL-404, and a rapid taper of methylprednisolone (125 mg to 30 mg daily). PBMCs were monitored by flow cytometry for B (CD20+) and T (CD3+) lymphocytes. KPL-404 levels were monitored at peak, trough, and longitudinally over time. Prior to transplantation, donor-specific IgM and IgG antibody levels were acceptably low.

Serial transthoracic echocardiography (TTE) using the Phillips EPIQ CVx Ultrasound system and X5-1 transducer was performed at least twice a week to closely monitor the xenograft. Endomyocardial biopsies (embx) of the right ventricle and right heart catheterization for pressures were planned for routine surveillance and as needed, permitted by clinical status. Histology was performed with Hematoxylin & Eosin (H&E) stains and immunohistochemistry (IHC) for CD3, CD20, CD68, CD3d and CD4d markers on embx specimens.

Serum levels of troponin I were followed longitudinally. Xenograft-derived cell free DNA (xd-cfDNA) were drawn weekly and determined off site by CareDx (Brisbane, Calif.).

Donor Specific Antibody (DSA) Assay

Heat-inactivated patient serum was incubated with 10 GE source animal aortic endothelial cells (pAEC) for 2 hrs at 4° C. Untreated pAECs were used as a negative assay control. Heat inactivated serum samples from healthy subjects known to have high (High Human) and low (Low Human) levels of anti-non-gal antibodies, served as controls. After incubation, pAEC were washed twice with PBS and non-specific protein-binding sites were blocked with 10% normal goat serum (Abcam, Mass., USA) for 20 min at 4° C. Then, fluorescence conjugated (FITC or Alexa-Fluor 488) goat anti-human IgM and IgG (Jackson ImmunoResearch Laboratories, PA, USA) was added (1:100 final concentration in PBS) and allowed to incubate for 60 min at 4° C. After incubation, the pAECs were washed twice with PBS and resuspended to perform Flow Cytometry (BD Accuri C6 Plus, CA, USA). Ten thousand events per sample were counted and samples were analyzed using FlowJo software (FlowJo LLC, OR, USA).

### Results

The organ transplant was successful, and the subject recovered from the surgery and was observed to have no signs of xenotransplant hyperacute rejection or acute rejection three days after surgery.

Following the surgery, various analyses were performed. As a result of changing dynamics in the subject after surgery, the dosing protocol relative to Tables 4-6 shown above was altered before, during, and after the transplant.

During the transplant surgery, blood loss was estimated to be >3 liters (KPL-404 was administered after hemostasis). Intraoperatively, cell saver 1800 cc (washed packed red cells) were administered. Blood products that were infused included 8 units of PRBCs, 4 units of platelets, 6 units FFP (with extra C1 Esterase Inhibitor (Berinert)) to account for infused complement), 341 cc Cryo, 24 mcg desmopressin (DDAVP®), and 2226 mg fibrinogen (Table 7 and 8).

TABLE 7
Intraoperative Fluid Input

| | | |
|---|---|---|
| Total IV fluid and | 6600 | mL |
| medication volume: | | |
| PRBC: | 8 | units (2400mL) |
| FFP: | 6 | units (1117 mL) |
| Platelets: | 4 | Units (600 mL) |
| Cryoprecipitate: | 341 | mL |
| Perfusion Input | 1249 | mL |
| Cellsaver | 1690 | mL |
| Total Input | 13,997 | mL |

TABLE 8
Intraoperative Fluid Output

| | | |
|---|---|---|
| Cellsaver EBL | 3380 | mL |
| Ultrafiltration on CPB | 4200 | mL |
| Urine Output | 650 | mL |
| Other Blood Loss and | 2000 | mL |
| insensible losses | | |
| Total Output | 10,230 | mL |

Net Output was 3,767 mL (13,997 mL total input—10,230 mL total output).

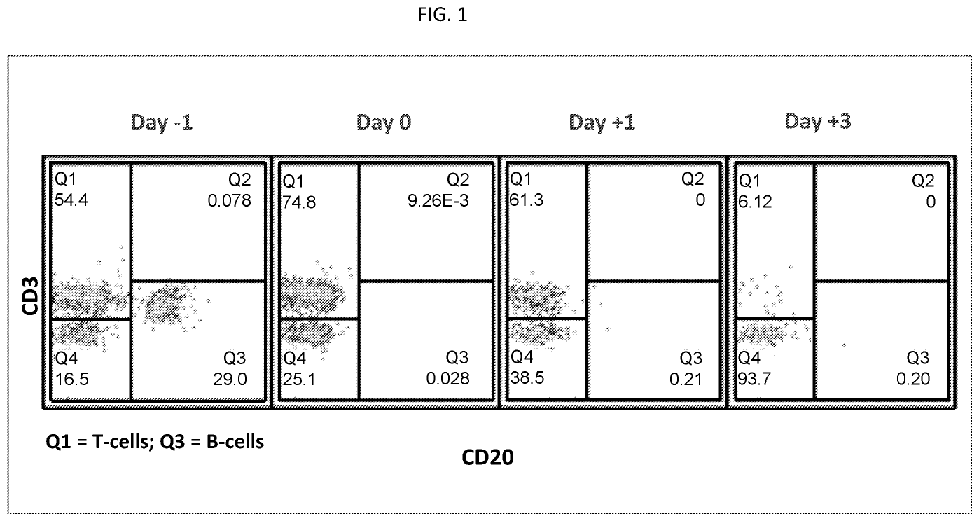

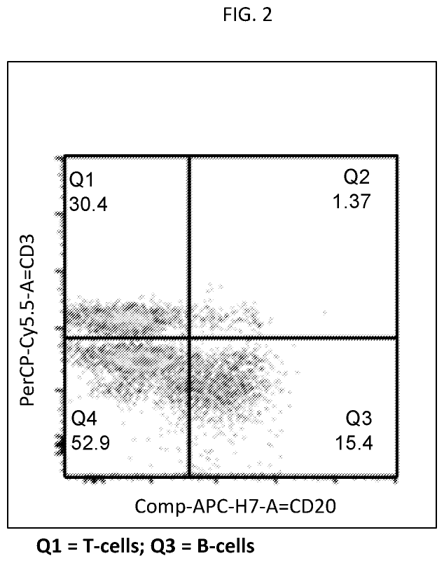

About 4-5 liters of crystalloid was used to resuspend and re-infuse the lost recaptured/red cells intraoperatively/perioperatively. Additional units of exogenous PRBC were given as intraoperative and post-operative transfusions, including 1 unit cryoprecipitate (Spared FFP status post (s/p) extra C1 Esterase Inhibitor (Berinert)), 1 unit pRBC, and 5 units platelets. B-cell counts and T-cell counts (and/or lymphocyte counts), both pre-operatively and post-operatively were measured from day −1 to day 7 (FIG. 1). The following dosages indicate the cumulative dose of the respective drugs administered to the subject at the indicated time of blood sample collection for flow cytometry: Day −1: prior to induction; Day 0: s/p Total: Rituximab 375 mg/m²; Day 1: s/p Total: Rituximab 375mg/m² and s/p 1 mg/kg anti-thymocyte globulin; Day 3: s/p Rituximab 375 mg/m², s/p 2 mg/kg anti-thymocyte globulin. Flow cytometry analysis of the peripheral bloodstream indicated depletion of B cells by Day 0 (see quadrant 3) and depletion of T cells by Day 3 (see quadrant 1). Notably, T cells were still present immediately following surgery, as shown in quadrant 1 of FIG. 1 on Day 0 and Day 1 as, in this case, ATG was not administered until after surgery (day 1). While B-cells were largely ablated in the periphery, other sources of CD40 exist that could contribute to transplant rejection. CD40 is present on several other cell-types, including antigen presenting cells (APCs), which could contribute to T-cell activation and transplant rejection. Furthermore, platelet-derived CD154 (CD4OL) release as a result of surgery may be sufficient to initiate transplant rejection independent of any cellular source of this molecule (Xu et al., J. Clin. Invest. 2006; 116(3):769-774). CD154 is a cell surface molecule expressed on activated T-cells that binds CD40, an activating molecule on APCs, which are believed to promote graft rejection. Together, this supports the role for CD40 blockade to reduce the risk of transplant rejection even in a subject who has undergone ablation of peripheral B-cells and/or T-cells. The absence of transplant rejection in the treated subject, in particular, hyperacute and acute rejection, demonstrates the effectiveness of KPL-404 in mediating CD40 blockade and mitigating against transplant rejection.

Flow cytometric analysis was performed on lymph nodes that were removed from the subject on the day the subject's chest was closed (day 2) following the heart transplant. At the time of chest closure, the cumulative dose of the respective drugs administered to the subject were as follows: s/p Total: Rituximab 375 mg/m², s/p 2 mg/kg anti-thymocyte globulin (FIG. 2). Notably, flow cytometry analysis shows the presence of B-cells (quadrant 3) and T-cells (quadrant 1) in the lymph nodes immediately before chest closure on Day 2, suggesting that (as expected) the rituximab and anti-thymocyte globulin did not fully deplete the B-cells or T-cells in solid tissue, even though the cells were depleted in the bloodstream (FIG. 1).

Rituximab and anti-thymocyte globulin were given relative to KPL-404 in the following regimen:
Day −1 WBC 3, Plt 90-100 CD3: not collected
Day 0 WBC 3.2, Plt 65; coming out of operating room; s/p rituximab and KPL-404 Day −1
Day 1 WBC 5.8, Plt 78; 1 mg/kg anti-thymocyte globulin, started MMF 500 mg BID
Day 2 WBC 12.6, Plt 74; 1 mg/kg anti-thymocyte globulin after chest closure, continued MMF and Steroid wean per schedule, CD3 23 (absolute), CD4 17 (absolute), CD8 <10
Day 3 WBC 16.9, Plt 93 . . . 2 mg/kg anti-thymocyte globulin after hemostasis s/p chest closure and reassuring platelets, CD3 absolute <10, CD4 absolute <10, CD8 <10
   For KPL-404 preparation, nine vials for each dose were used, drawn up 8.5 ml for 850 mg (10 mg/kg). Heparin was given on Day 0 (30,000 units Bolus) and 250 mg protamine was administered prior to leaving operating room. Running partial thromboplastin time (PTT) was 45-55 seconds. 1.5× ULN was administered for the last 3 days with Bivalrudin ˜0.06 mcg/kg/min. Extracorporeal membrane oxygenation (ECMO) was removed on Day 4.

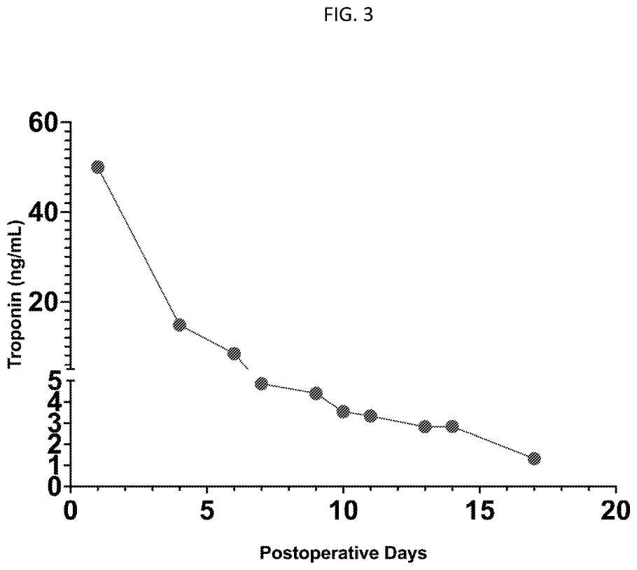

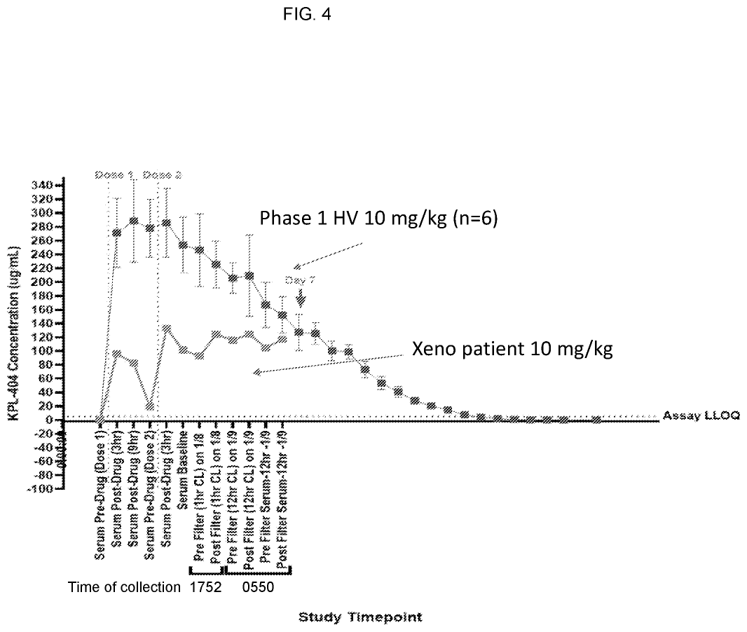

Following transplantation, troponin levels were measured in the subject (FIG. 3). Troponin is a biomarker for transplant rejection and is released by the heart's myocytes in response to injury. The subject exhibited an increase in serum troponin I levels postoperatively, but these levels decreased to baseline by POD24 suggesting that the heart recovered from the surgical procedure and that no transplant rejection occurred following the transplantation procedure. Troponin I levels began increasing by POD 35, although it was later determined by endomyocardial biopsy (POD 50) that this did not appear to be a result of rejection, as there were no signs of antibody or acute cellular rejection (International Society for Heart and Lung Transplantation (ISHLT) Grade 0).

Left ventricular ejection fraction (LVEF) remained normal or hyperdynamic throughout the postoperative course. Left ventricular (LV) and right ventricular (RV) thickness, LV chamber size (LV end diastolic dimension), and global longitudinal strain (GLS) changed. LV thickness measured between 1.2-1.4 cm along the basal interventricular septum and posterior wall, while the RV measured 1.0-1.1 cm on short axis views. GLS was noted to be very compliant at baseline, with GLS scores ranging from −15 to −22 without contrast.

On POD 34, embx demonstrated no evidence of rejection and the heart revealed a right atrial pressure of 5 mmHg, pulmonary artery pressure of 25/15 mmHg, cardiac index of 2.7 (L/min/BSA) and a mixed venous saturation of 65% (Hgb 8.1 gm/dL). The patient was able to rehabilitate without any cardiovascular support and the xenograft functioned normally without evidence of rejection.

On POD 43 he became more somnolent, was intubated and developed hypotension, which was responsive to fluid and vasopressin administration. His chest radiograph suggested worsening left lower lobe collapse and soft infiltrates in the right lung. Bronchoscopy revealed diffuse shallow ulcers throughout the right primary and secondary airways suggestive of viral or fungal etiology, despite ongoing prophylaxis. Given bronchoscopic findings, clinical decline suggestive of sepsis, and profound hypogammaglobulinemia (total IgG 185 mg/dL), antimicrobial coverage was broadened and the patient was administered 1 gm/kg IVIg (80 gm). Anti-viral therapy was changed from ganciclovir to cidofovir. A biopsy of these airway lesions was performed which did not demonstrate any viral cytopathic effect or viral inclusions and GMS stains were negative. A repeat bronchoscopy performed approximately 5 days later showed improvement in the diffuse airway ulcerations. The patient was ultimately extubated and resumed in-room rehabilitation.

On POD 49, the patient's serum lactate rose from 4 mg/dL to 11.2 mg/dL. He became hypotensive, required vasopressors and was intubated. He developed acrocyanosis suggesting a lowered cardiac output for the first time since transplant. He underwent a negative exploratory laparotomy. A newly placed pulmonary artery catheter revealed a mixed venous saturation of 33%. While echocardiogram revealed an EF of 65-70%, a dramatic increase in LV wall thickness (1.7 cm) was observed and RV thickness (1.4 cm) was noted. Both LV and RV walls remained persistently thickened, independent of left ventricular end diastolic volume and loading conditions. GLS became dramatically more positive. He was re-cannulated for a life supporting run of VA-ECMO.

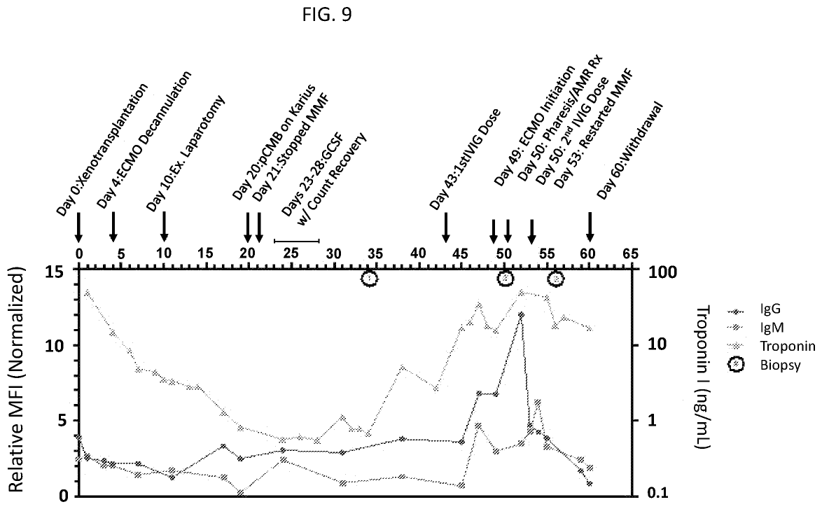

Endomyocardial biopsy (POD 50) did not demonstrate antibody or acute cellular rejection (International Society for Heart and Lung Transplantation (ISHLT) Grade 0). There was focal capillary damage with extravasated erythrocytes and edema. Antibody staining, IgG greater than IgM, in capillaries was present but was negative for C3d or C4d. There was a single ischemic myocyte and no cellular infiltrates either on H&E or IHC by CD3+ or CD68+ staining. Troponin I was rising (FIG. 9) and it was also determined that xd-cfDNA and serum levels of xenograft-specific IgG, and to lesser extent, IgM were peaking. An atypical presentation of antibody mediated rejection was suspected, so initiated treatment by plasmapheresis (over 5 days, first at 1.5× plasma volume exchange, followed by 3 additional sessions with 1.0× plasma volume exchanges), IVIg 1 gm/kg (60 gm), complement inhibition (Berinert x2, Eculizumab x1) and B-cell depletion (Rituximab 375 mg/m2). The patient was continued on ECMO support.

On Day 56 repeat embx revealed ISHLT pAMR1 (improved capillary staining of IgG and IgM, but C4d staining was now present). There was interval reduction in interstitial red blood cell extravasation and edema, but embx revealed 40% myocyte necrosis. Again, there was no evidence of cellular rejection, but C4d, IgG, and IgM were weakly positive and more prominent in the increased areas (40%) of necrotic myocytes, which may have indicated non-specific binding. There was focal capillary damage with extravasated erythrocytes and stromal cells within the prominent inter-myocyte edema. Repeat echocardiogram demonstrated an EF of >70%, normal RV function and improved longitudinal strain (−19.2). Biventricular wall thickening improved slightly. The patient was slowly able to be weaned from 4.5 L/min of VA-ECMO flows down to 3 L/min without the need for catecholamine support. Further echocardiographs did not demonstrate improvement of wall thickness or GLS and ECMO could not be weaned below 2 L/min.

An irreversible injury to the xenograft was observed and support was compassionately withdrawn. Preliminary postmortem exam of the heart revealed an increase in heart weight to 600 gm, up from 328 gm at transplantation. Cardiac myocytes were widely spaced with central nuclei separated by thin bands of fibrosis. Necrosis of myocytes was scattered and observed with loss of myocyte integrity. Small caliber vessels demonstrated prominent endothelial nuclei. Erythrocytes were observed scattered throughout the interstitial space between myocytes in a pattern consistent with vascular extravasation. Additional studies are underway to characterize the pathophysiologic mechanisms resulting in this damage.

### KPL-404 Serum Concentration Measurement

KPL-404 blood serum concentration was measured following administration of the antibody (FIG. 4). Following dose 1 (part 1 of the first induction dose), the blood serum concentration reached maximum (peak) concentration of about 100 μg/mL and a minimum (trough) concentration of ˜20 μg/mL before a subsequent second dose (part 2 of the first induction dose) was administered. This contrasts with previous phase 1 studies in which the normalized blood serum concentration was over 250 μg/mL at a similar time point.

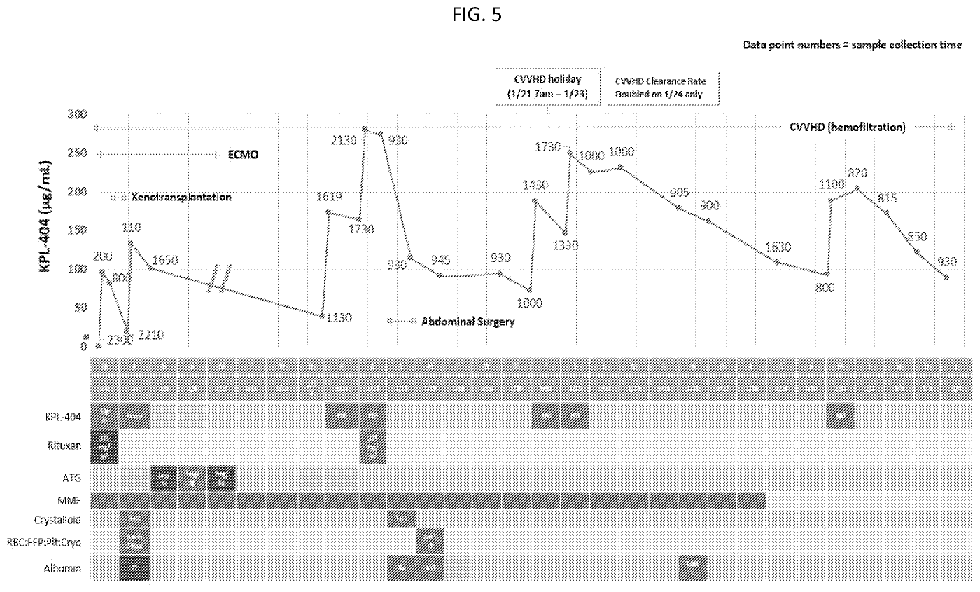

An extended time course is shown in FIG. 5, illustrating a minimum KPL-404 concentration of 20 μg/mL was sufficient to prevent transplant rejection.

The serum concentration of KPL-404 was determined using an immunoassay. The KPL-404 immunoassay employed plates coated with an anti-ID antibody as the capture antigen. Calibration standards and quality control (QC) samples are prepared by diluting stock solutions of KPL-404 into undiluted human serum, followed by subsequent serial dilution with undiluted human serum to the desired KPL-404 concentration. Prepared calibrators, quality control samples and study human serum samples were diluted to a minimum required dilution (MRD) of 1:200 using Assay Diluent prior to loading onto the pre-coated plates. The plates were incubated to allow the KPL-404 present in the samples to bind to the target and subsequently washed to remove unbound material. Mouse anti-Hu IgG4 pFc HRP was then added to the plate to bind the KPL-404. The plates were further incubated, and then washed to remove any unbound materials. TMB substrate was then added to the plates and then the reaction was stopped by adding 1N H₂SO₄. The plate was read immediately using a SPECTRAMAX® Plus 384 (450 nm for detection and 650 nm for background) microplate reader. The resulting OD values (detection OD at 450 nm minus background OD at 650 nm) obtained from the calibration standards were fitted using a 4-PL logistic equation with 1/y2 to calculate the KPL-404 concentrations in the quality control and study samples.

The calibration curve range of this method was 8000 ng/mL to 80.0 ng/mL in 100% human serum. Calibrators outside the evaluated range of the assay at 10000 ng/mL and 40.0 ng/mL in 100% human serum may be included as anchor points to facilitate curve-fitting.

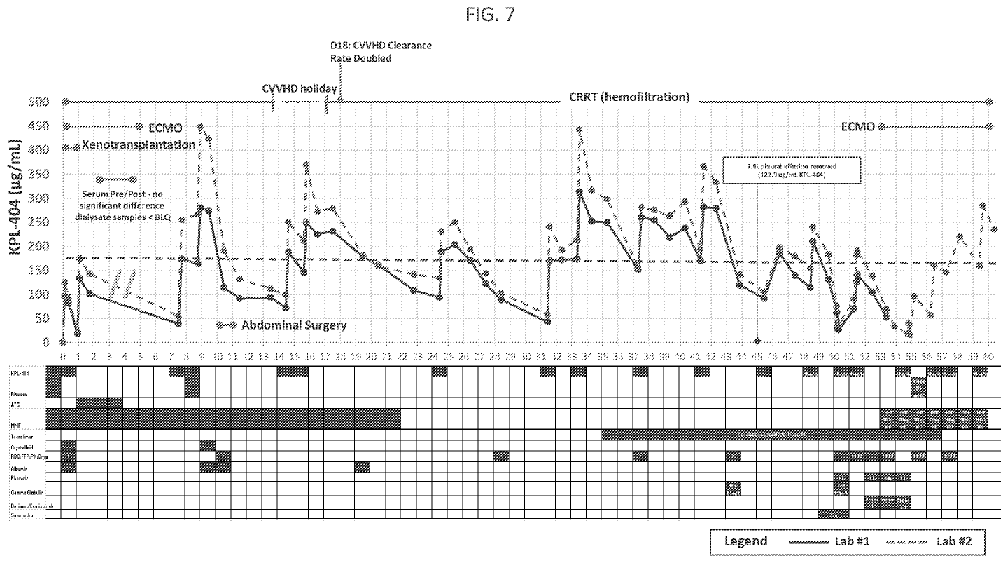

An updated extended time course over 60 days is shown in FIG. 7, and no signs of transplant rejection were observed in the subject at this time. A time course of therapeutic administrations corresponding to the times shown in FIG. 7 is provided in Table 9.

TABLE 9
Time course of Therapeutic Administrations

| | | | | | | | RBC: | | | | | |
|---|---|---|---|---|---|---|---|---|---|---|---|---|
| Post- | | | | | | | FFP: | | | Gamma | | |
| operative | KPL- | | | | | | Plt: | | | Globulin | Berinert/ | |
| Days | 404 | Rituxan | ATG | MMF | Tacrolimus | Crystalloid | Cryo | Albumin | Pheresis | (IVIg) | Eculizumab | Solumedrol |
| −1 | X | X | | X | | | | | | | | |
| Day of | X | | | X | | X | X | X | | | | |
| Surgery | | | | | | | | | | | | |
| 1 | | | X | X | | | | | | | | |
| 2 | | | X | X | | | | | | | | |
| 3 | | | X | X | | | | | | | | |
| 4 | | | | X | | | | | | | | |
| 5 | | | | X | | | | | | | | |
| 6 | | | | X | | | | | | | | |
| 7 | X | | | X | | | | | | | | |
| 8 | X | X | | X | | | | | | | | |
| 9 | | | | X | | X | | X | | | | |
| 10 | | | | X | | | X | X | | | | |
| 11 | | | | X | | | | | | | | |
| 12 | | | | X | | | | | | | | |
| 13 | | | | X | | | | | | | | |
| 14 | X | | | X | | | | | | | | |
| 15 | X | | | X | | | | | | | | |
| 16 | | | | X | | | | | | | | |
| 17 | | | | X | | | | | | | | |
| 18 | | | | X | | | | | | | | |
| 19 | | | | X | | | | X | | | | |
| 20 | | | | X | | | | | | | | |
| 21 | | | | X | | | | | | | | |
| 22 | | | | | | | | | | | | |
| 23 | | | | | | | | | | | | |
| 24 | X | | | | | | | | | | | |
| 25 | | | | | | | | | | | | |
| 26 | | | | | | | | | | | | |
| 27 | | | | | | | | | | | | |
| 28 | | | | | | | X | | | | | |
| 29 | | | | | | | | | | | | |
| 30 | | | | | | | | | | | | |
| 31 | X | | | | | | | | | | | |
| 32 | | | | | | | | | | | | |
| 33 | X | | | | | | | | | | | |
| 34 | | | | | | | | | | | | |
| 35 | | | | | Tacro | | | | | | | |
| 36 | | | | | Sublingual, | | | | | | | |
| 37 | X | | | | 1 gm BID, | | X | | | | | |
| 38 | | | | | Goal | | | | | | | |
| 39 | | | | | trough | | | | | | | |
| 40 | | | | | 3-5 | | | | | | | |
| 41 | X | | | | | | | | | | | |
| 42 | | | | | | | | | | | | |
| 43 | | | | | | | X | | | 80 g | | |
| 44 | | | | | | | | | | | | |
| 45 | X | | | | | | | | | | | |
| 46 | | | | | | | | | | | | |
| 47 | | | | | | | | | | | | |
| 48 | X | | | | | | | | | | | |
| 49 | | | | | | | | | | | | 3 gm |
| 50 | X | | | | | | 4:4:4:3 | | 1.5x | 60 g | | |
| 51 | X | | | | | | | | | | | |
| 52 | | | | | | | | | 1.0x | | Berinert | |
| 53 | | | | MMF | | | 2:0:0:0 | | 1.0x | | Berinert | |
| | | | | 500 mg | | | | | | | | |
| | | | | BID | | | | | | | | |
| 54 | X | | | MMF | | | | | 1.0x | | Eculizumab | |
| | | | | 500 mg | | | | | | | | |
| | | | | BID | | | | | | | | |
| 55 | | X | | MMF | | | 4:0:4:0 | | | | | |
| | | | | 500 mg | | | | | | | | |
| | | | | BID | | | | | | | | |
| 56 | X | | | MMF | | | | | | | | |
| | | | | 500 mg | | | | | | | | |
| | | | | BID | | | | | | | | |
| 57 | X | | | MMF | | | 1:0:0:0 | | | | | |
| | | | | 500 mg | | | | | | | | |
| | | | | BID | | | | | | | | |
| 58 | | | | MMF | | | | | | | | |
| | | | | 500 mg | | | | | | | | |
| | | | | BID | | | | | | | | |
| 59 | X | | | MMF | | | | | | | | |
| | | | | 500 mg | | | | | | | | |
| | | | | BID | | | | | | | | |

The dotted line in FIG. 7 corresponds to the original calculation of concentration of KPL-404 (e.g., as shown in FIG. 5). Due to minor inconsistencies in the assay, these samples were re-run using the same protocol but with different machines. The result of the re-analysis is shown by the solid line. The overall trend is similar between the dotted and solid lines, but with slight variation of the peaks. One hypothesis for the discrepancy may be that the original assay was less sensitive and, thus, resulted in a higher readout than was actually present.

As is shown in FIG. 7, at Day 44 the subject was treated by thoracentesis to remove a pleural effusion, which led to a drop in the KPL-404 antibody levels to ˜100 μg/mL. 1.6 L of pleural effusion was collected from the lungs of the subject. The concentration of KPL-404 present in the pleural effusion was, unexpectedly, approximately the same as concentration present in the subject's plasma. At days 51, 53, and 54, the subject underwent plasmapheresis, which separated the plasma from the remainder of his blood components. The plasma was removed, thus leading to a drop in KPL-404 antibody levels to ˜50-75 μg/mL.

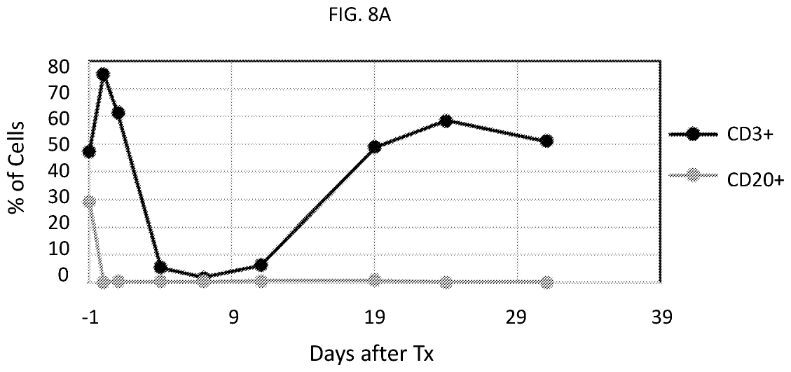

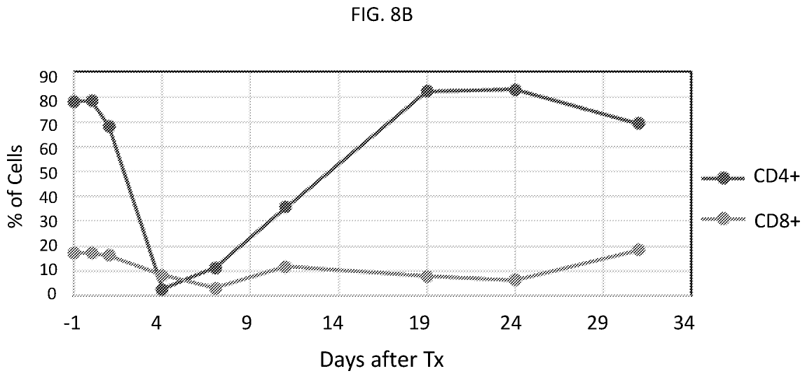

B cell and T cell counts were assessed following the transplant. FIGS. 8A and 8B show percentages of B cells and T cells counts in peripheral blood relative to total blood lymphocyte count. These graphs indicate that T cells were still present in the subject at the time of transplant and T-cell count steadily increased over time soon after transplant. By contrast, B cell counts remained low after transplant, possibly due to the action of the B-cell depleting antibody, rituximab. The use of a CD40-based immunomodulation regimen and genetically modified xenograft avoided hyperacute rejection in the immediate postoperative period. Moreover, endomyocardial biopsies of the xenograft did not demonstrate acute cellular rejection. Antibody mediated rejection was not detected by ISHLT criteria, with the exception of mild C4d staining POD 56 (which includes IgM/IgG staining and C4d staining of intact myocardium, monocellular infiltrate and endotheliitis with myocardial thickening from fibrosis). In non-human primate studies, microthrombi, massive intracellular hemorrhage, myocyte necrosis with pyknotic nuclei and occasional cellular infiltrate are generally present in xenografts with antibody mediated rejection, which was absent in this case. No evidence of pathology outside of the heart related to the xenotransplantation and associated therapeutics were observed on preliminary autopsy.

IVIg was administered twice during the patient's postoperative course (FIG. 7 and Table 9). The first dose on POD 43 for infectious concerns. The second dose, on POD 50 for treatment of presumed antibody mediated rejection after initial therapeutic plasma exchange. Notably, both doses coincided with an increase in recipient donor-specific IgG and to a lesser extent IgM. It is unclear at this time whether IVIg contributed to xenograft capillary injury. In a single report, IVIg has not shown to have complement dependent cytotoxicity to pig xenogeneic tissues in vitro or in vivo (Yamamoto et al., Sci Rep 2020; 10(1)11747). However, binding to donor specific cells was observed in two laboratories, including ours. In the future, should IVIg be deemed necessary, lots should be tested to select those that exhibit minimal xenograft specific antibody-mediated cytotoxicity.

Postoperative monitoring of both the xenograft and zoonoses were performed using traditional clinical monitoring and included novel highly sensitive cfDNA assays. Longitudinal xenograft-specific antibody assays were also used to aid in detecting elicited donor-specific antibody responses. These results were generally delayed by 1 week, as they are dependent on growing cell cultures of the source animal's aortic endothelial cells, incubating these cells with recipient serum and analyzing antibody binding by flow cytometry. Point-of-care troponin I testing was most useful for detecting early xenograft injury largely because the data was instantaneous and previously validated by NHP work (FIG. 9).

The donor spleen tested positive for pCMV indicating that the donor was likely latently infected with pCMV. Recipient PBMCs were also positive. It is uncertain whether the detection of pCMV via plasma mcf-DNA and/or PCR testing represents 1) replicating virus in the xenograft or 2) replicating virus in the recipient or 3) shedding of genetic material from the xenograft. The presence of pCMV in explanted xenografts from NHP recipients has been correlated with worse outcomes than those without pCMV, for reasons that are currently unclear (Denner et al., Sci Rep 2020; 10(1)1 7531). Further viral testing is warranted since human herpes virus 6 (HHV6) was also detected in lung lavage of this patient. HHV6 has been shown to cross-react with pCMV and where HHV6 is a significant cause of allograft rejection (Gu et al., Virology 2014; 460-461:165-72; and Fiebig et al., Viruses 2017; 9(11):317).

### Example 3

A human subject is selected for an allogeneic liver transplant. The subject may be administered a therapeutic regimen (e.g., induction therapy) before the transplant (e.g., within 1-24 hours before the transplant) that includes 125 mg methylprednisolone, 10 mg/kg rituximab, 20 U/kg C1 esterase inhibitor and 10 mg/kg of KPL-404.

The subject undergoes the transplant and may receive 1000 mg methylprednisolone during the operation. Once the subject reaches hemostasis following the transplant, the subject may also receive a therapeutic regimen (e.g., maintenance therapy) that includes 10 mg/kg KPL-404, 20 U/kg C1 esterase inhibitor, and 2 mg/kg anti-thymocyte globulin.

The next day, 24 hours after the surgery, the subject may receive an additional therapeutic regimen that includes 20 U/kg C1 esterase inhibitor and 2 mg/kg anti-thymocyte globulin.

Starting one week after the transplant, the subject may receive a periodic intravenous infusion of KPL-404 at a dosage of 10 mg/kg (e.g., at least one dose every two or three weeks). The blood of the subject is periodically assayed to measure his blood serum concentration of KPL-404. When the subject's blood serum concentration of KPL-404 is within 10% of 150 μg/mL (i.e., drops below 165 μg/mL), a maintenance dose of KPL-404 may be intravenously administered to boost the subject's KPL-404 blood serum concentration. The time between maintenance doses may be determined empirically based on the PK profile of the subject and the peak and trough values of KPL-404.

### Example 4

A human subject is selected for a xenograft kidney transplant. Kidney transplantation can include induction therapy with a biologic lymphocyte-depleting agent begun before, at the time of, or immediately after transplantation. For example, the subject may be treated with an immunosuppressive standard of care as described, e.g., in Yin et al. Chin. Med. J. 124:1928-1932, 2011, or Amer. J. Transplantation, 9: Suppl 3, 2009, S1-155, each of which is hereby incorporated by reference in its entirety, which includes rituximab and anti-thymocyte globulin therapy. The subject may be administered a therapeutic regimen (e.g., induction therapy) before the transplant (e.g., within 1-24 hours before the transplant) that includes a B cell and/or T-cell depleting agent. After administration of the B cell depleting agent or T-cell depleting agent, the subject may be subcutaneously or intravenously administered a first induction dose (i.e., a conditioning dose) of between 1 and 20 mg/kg (e.g., 5 mg/kg or 10 mg/kg) of KPL-404. The subject may be administered one or more additional agents during the transplant (e.g., methylprednisolone (e.g., ˜1000 mg)) during the operation.

Following the transplant, and once the subject reaches hemostasis, the subject may also subcutaneously or intravenously be administered a second dose (e.g., an induction dose) of KPL-404 of between 1 and 20 mg/kg (e.g., 5 mg/kg or 10 mg/kg), and, optionally, 20 U/kg C1 esterase inhibitor and/or a T cell depleting agent.

The next day, 24 hours after the surgery, the subject may optionally receive an additional therapeutic regimen that includes a T cell depleting agent.

The subject may also receive intravenous or subcutaneous administration of a second induction dose of KPL-404, as either a divided dose of at least two doses of between 1 and 20 mg/kg (e.g., 5 mg/kg or 10 mg/kg per dose) administered 1 to 24 hours apart, or as a single, undivided dose of between 1 and 20 mg/kg (e.g., 5 mg/kg or 10 mg/kg per dose)) that is administered between 2-10 days after surgery. Until the PK of KPL-404 is replicable or predictable in the subject (as determined by periodic measurement of KPL-404 concentration in the subject's blood, plasma, or serum), additional induction doses of KPL-404 are delivered to the subject as needed to maintain C_{trough} of KPL-404 at or above therapeutic levels.

Once the PK of KPL-404 in the subject's blood, plasma, or serum is replicable or predictable, the subject may receive a periodic intravenous infusion of KPL-404 at a dosage of 10 mg/kg (i.e., a maintenance dose, e.g., at least one dose every one, two or three weeks). Optionally, the blood of the subject is periodically assayed to measure the blood, plasma, or serum concentration of KPL-404. When the subject's blood, plasma, or serum concentration of KPL-404 is within 10% of 10 μg/mL (i.e., drops below 11 μg/mL), a maintenance dose of KPL-404 may be intravenously administered to boost the subject's KPL-404 blood, plasma, or serum concentration. The optimal time between maintenance doses may be determined empirically based on the PK profile of the subject and the peak and trough values of KPL-404.

The subject's troponin levels can be periodically measured to determine a level of transplant rejection. If the troponin levels increase, the subject can be administered an infusion of KPL-404 or an increased dosage of KPL-404 as further described herein.

### Other Embodiments

While specific aspects of the invention have been described and illustrated, such aspects should be considered illustrative of the invention only and not as limiting the invention as construed in accordance with the accompanying claims.

All publications and patent applications cited in this specification are herein incorporated by reference in their entirety for all purposes as if each individual publication or patent application were specifically and individually indicated to be incorporated by reference for all purposes. Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

1. A method of treating or reducing a risk of transplant rejection or increasing a duration of time before transplant rejection occurs in a human subject receiving or having received a transplant comprising administering an anti-CD40 antibody or antigen-binding fragment thereof from about 10 hours to about 24 hours prior to the transplant and administering the anti-CD40 antibody or antigen-binding fragment thereof within about 1 hour to about 24 hours following the transplant and once the subject achieves hemostasis, wherein the antibody or antigen-binding fragment thereof is administered subcutaneously or intravenously.

2. The method of claim 1, wherein the antibody or antigen-binding fragment thereof comprises a heavy chain variable region comprising a CDRH1, CDRH2, and CDRH3 as set forth SEQ ID NOs: 1-3, respectively, and a light chain variable region comprising a CDRL1, CDRL2, and CDRL3 as set forth in SEQ ID NOs: 4-6, respectively.

3. (canceled)

4. A method of treating or reducing a risk of transplant rejection or increasing a duration of time before transplant rejection occurs in a human subject receiving or having received a transplant comprising administering a humanized anti-CD40 antibody or antigen-binding fragment thereof comprising a heavy chain variable region comprising a CDRH1, CDRH2, and CDRH3 as set forth SEQ ID NOs: 1-3, respectively, and a light chain variable region comprising a CDRL1, CDRL2, and CDRL3 as set forth in SEQ ID NOs: 4-6, respectively, and wherein the antibody or antigen-binding fragment thereof is administered subcutaneously or intravenously at a dosage of about 1 mg/kg to about 20 mg/kg.

5. The method of claim 1, wherein the heavy chain variable region comprises an amino acid sequence having at least 85% sequence identity to SEQ ID NO: 7, and wherein the light chain variable region comprises an amino acid sequence having at least 85% sequence identity to SEQ ID NO: b 8.

6. The method of claim 5, wherein the heavy chain variable region comprises the amino acid sequence set forth in SEQ ID NO: 7, and wherein the light chain variable region comprises the amino acid sequence set forth in SEQ ID NO: 8, or wherein the heavy chain comprises the amino acid sequence set forth in SEQ ID NO: 9, and wherein the light chain ocmprises the amino acid sequence set forth in SEQ ID NO:b 10.

7. (canceled)

8. The method of claim 4, wherein the antibody or antigen-binding fragment thereof is administered prior to the transplant on the same day as the transplant, and/or after the transplant.

9. The method of claim 8, wherein the antibody or antigen-binding fragment thereof is administered:
(a) from about 10 hours to about 24 hours prior to the transplant; and/or
(b) with about 1 hour to about 24 hours following the transplant and once the subject achieve hemostasis.

10.-12. (canceled)

13. The method of claim 9, wherein the antibody or antigen-binding fragment thereof is administered:
(a) one day, two days, three days, four days, five days, six days, one week, two weeks, three weeks, four weeks, two months, three months, four months, five months, six months, seven months, eight months, nine months, ten months, 11 months, and/or one year after the transplant;
(b) for a treatment period of one day, two days, three days, four days, five days, six days, one week, two weeks, three weeks, four weeks, two months, three months, four months, five months, six months, seven months, eight months, nine months, ten months, eleven months, one year, two years, three years, four years, five years, six years, seven years, eight years, nine years, ten years, or for the life of the subject; and/or
(c) at least once every week, once every two weeks, once every three weeks, or monthly for a treatment period of one week, two weeks, three weeks four weeks, two months, three months, four months, five months, six months, seven months, eight months, nine months, ten months, eleven months, one year, two years, three years, four years, five years, six years, seven years, eight years, nine years, ten years, or for the life of the subject.

14.-15. (canceled)

16. The method of claim 1, further comprising administering a first therapeutic agent that depletes or reduces B cells in the subject.

17. The method of claim 16, wherein the first therapeutic agent is administered
(a) prior to the transplant;
(b) after the transplant;
(c) for a treatment period of on day, two days, three days, four days, five days, six days, one week, two weeks, three weeks, or four weeks;
(d) daily, weekly, or once every other week for a treatment period of one week, two weeks, three weeks, or four weeks;
(g) at a dosage of about 1 mg/kg to about 40 mg/kg: and/or;
(f) intravenously.

18. The method of claim 17, wherein the first therapeutic agent is administered from about 10 hours to about 24 hours prior to the transplant and/or one day, two days, three days, four days, five days, six days, one week, two weeks, three weeks, and/or four weeks after the transplant.

19-25. (canceled)

26. The method of claim 16, wherein the first therapeutic agent is rituximab.

27. The method of claim 26, wherein the rituximab is administered at a dosage of about 250 mg/m² to about 500 mg/m².

28. (canceled)

29. The method of claim 1, further comprising administering a second therapeutic agent that depletes or reduces T cells in the subject.

30. The method of claim 29, wherein the second therapeutic agent is administered:
(a) prior to the transplant;
(b) on the same day as the transplant;
(c) after the transplant;
(d) for a treatment period of one day, two days, three days, four days, five days, six days, one week, two weeks, three weeks, or four weeks;
(e) daily, weekly, or once every other week for a treatment period of one week, two weeks, three weeks, or four weeks;
(f) at a dosage of about 1 mg/kg to about 10 mg/kg; and/or
(g) intravenously.

31. The method of claim 29, wherein the second therapeutic agent is administered:
(a) from about 10 hours to about 24 hours prior to the transplant;
(b) administered within about 1 hour to about 24 hours following the transplant and once the subject achieves hemostasis; and/or
(c) one day, two days, three days, four days, five days, six days, one week, two weeks, three weeks, and/or four weeks after the transplant.

32-40. (canceled)

41. The method of any one of claims 29, wherein the second therapeutic agent is anti-thymocyte globulin.

42. The method of any one of claims 1, wherein the antibody or antigen-binding fragment thereof is administered at a dosage of about 10 mg/kg.

43. The method of any one of claims 1, wherein the transplant:
(a) comprises a heart, kidney, lung, liver, pancreas, intestine, thymus, skin, eye, uterus, stem cell, bone, tendon, cornea, heart valve, nerve, vein, or a portion thereof;
(b) is an allogeneic transplant; or
(c) is a xenograft transplant.

44-45. (canceled)

46. The method of claim 43, wherein the xenograft transplant comprises a cell, tissue, or organ or portion thereof from a pig, a cow, a horse, a dog, a cat, a sheep, a goat, or a non-human primate.

47. The method of claim 43, wherein the xenograft transplant comprises a porcine heart and/or comprises a cell, tissue, or organ or portion thereof from a host animal that has been genetically engineered to add, remove, or modify one or more xenoreactive antigens.

48. (canceled)

49. The method of any one of claims 1, further comprising administering one or more of a steroid, an antihistamine, an H2 receptor blocker, an antiviral agent, a complement inhibitor, an immunosuppressive agent, an anti-inflammatory agent, an anticoagulant, and an antibiotic.

50-69. (canceled)

70. A method of treating or reducing a risk of transplant rejection or increasing a duration of time before transplant rejection occurs in a human subject receiving or having received a transplant comprising:
(a) administering intravenously or subcutaneously an induction dose of an anti-CD40 antibody or antigen-binding fragment thereof, comprising a first dose of about 1 mg/kg to about 20 mg/kg from about 10 hours to about 24 hours prior to the transplant and a second dose of about 1 mg/kg to about 20 mg/kg, after the transplant once the subject achieves hemostasis; and
(b) administering subcutaneously or intravenously a maintenance dose of the anti-CD40 antibody or antigen-binding fragment thereof at a dosage of about 1 mg/kg to about 20 mg/kg for treatment period of at least one month.

71. The method of claim 70, wherein the maintenance dose is provided to maintain a minimum blood, plasma, or serum concentration of the anti-CD40 antibody or antigen-binding fragment thereof of at least 150 μg/ml.

72-99. (canceled)

100. A method of treating or reducing a risk of transplant rejection or increasing a duration of time before transplant rejection occurs in a human subject receiving or having received a transplant comprising:
(a) administering intravenously or subcutaneously a first induction dose of an anti-CD40 antibody or antigen-binding fragment thereof, comprising a first divided dose of about 1 mg/kg to about 20 mg/kg from about 1 hours to about 24 hours prior to the transplant and a second divided dose of about 1 mg/kg to about 20 mg/kg from within about 24 hours after the transplant once the subject achieves hemostasis, and a second induction dose comprising a first divided dose of about 1 mg/kg to about 20 mg/kg within about 1 day to about 14 days after the transplant once the subject achieves hemostasis and a second divided dose of about 1 mg/kg to about 20 mg/kg within about 1 hour to about 12 hours after the first divided dose of the second induction dose; and
(b) administering subcutaneously or intravenously a maintenance dose of the anti-CD40 antibody or antigen-binding fragment thereof at a dosage of about 1 mg/kg to about 20 mg/kg for treatment period of at least one month.

101. The method of claim 100, wherein the maintenance dose is provided to maintain a minimum blood, plasma, or serum concentration of the anti-CD40 antibody or antigen-binding fragment thereof of at least 10 μg/ml.

102. The method of claim 100, further comprising, prior to step (b), administering a third and/or fourth induction dose of about 1 mg/kg to about 20 mg/kg of the antibody or antigen-binding fragment thereof within about 1 day to about 14 days after the second induction dose.

103. (canceled)

104. A method of treating or reducing a risk of transplant rejection or increasing a duration of time before transplant rejection occurs in a human subject receiving or having received a transplant comprising:
(a) administering intravenously or subcutaneously an induction dose of an anti-CD40 antibody or antigen-binding fragment thereof, comprising a conditioning dose of about 1 mg/kg to about 20 mg/kg from about 10 hours to about 24 hours prior to the transplant and a first induction dose of about 1 mg/kg to about 20 mg/kg, at a time post-transplant and after hemostasis has been achieved; and
(b) administering subcutaneously or intravenously a maintenance dose of the anti-CD40 antibody or antigen-binding fragment thereof at a dosage of about 1 mg/kg to about 20 mg/kg for treatment period of at least one month.

105. The method of claim 104, further comprising, prior to step (b), administering at least one or more additional induction doses of about 1 mg/kg to about 20 mg/kg of the antibody or antigen-binding fragment thereof within about 1 day to about 14 days after the first induction dose.

106. The method of claim 105, wherein step (b) is initiated once the PK of the anti-CD40 antibody or antigen-binding fragment thereof is replicable or predictable.


SEQUENCE LISTING (10 sequences)

SEQ ID NO: 1 - protein, 9 aa, synthetic construct
YTFTNYWMH

SEQ ID NO: 2 - protein, 17 aa, synthetic construct
YINPSNDYTKYNQKFKD

SEQ ID NO: 3 - protein, 5 aa, synthetic construct
QGFPY

SEQ ID NO: 4 - protein, 10 aa, synthetic construct
SASSSVSYMH

SEQ ID NO: 5 - protein, 7 aa, synthetic construct
DTSKLAS

SEQ ID NO: 6 - protein, 9 aa, synthetic construct
HQLSSDPFT

SEQ ID NO: 7 - protein, 114 aa, synthetic construct
QVQLVQSGAEVKKPGASVKVSCKASGYTFTNYWMHWVRQAPGQRLEWIGYINPSNDYTKYNQKFKDRATLTADKSANTAYMELSSLRSEDTAVYYCARQGFPYWGQGTLVTVSS

SEQ ID NO: 8 - protein, 106 aa, synthetic construct
EIVLTQSPATLSLSPGERATLSCSASSSVSYMHWYQQKPGQAPRRWIYDTSKLASGVPARFSGSGSGTDYTLTISSLEPEDFAVYYCHQLSSDPFTFGGGTKVEIK

SEQ ID NO: 9 - protein, 441 aa, synthetic construct
QVQLVQSGAEVKKPGASVKVSCKASGYTFTNYWMHWVRQAPGQRLEWIGYINPSNDYTKYNQKFKDRATLTADKSANTAYMELSSLRSEDTAVYYCARQGFPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 10 - protein, 213 aa, synthetic construct
EIVLTQSPATLSLSPGERATLSCSASSSVSYMHWYQQKPGQAPRRWIYDTSKLASGVPARFSGSGSGTDYTLTISSLEPEDFAVYYCHQLSSDPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC